(19) United States
(12) Patent Application Publication
(10) Pub. No.: US 2023/0107522 A1
(43) Pub. Date: Apr. 6, 2023

(54) DATA REPOSITORY, SYSTEM, AND METHOD FOR COHORT SELECTION

(71) Applicant: GUARDANT HEALTH, INC., Palo Alto, CA (US)

(72) Inventors: Naveen KUMAR, Redwood City, CA (US); Jingwen ZHANG, Killeen, TX (US); Nisha SUBRAMANIAN, Redwood City, CA (US); Gautam NAYAK, Fremont, CA (US)

(21) Appl. No.: 17/823,915

(22) Filed: Aug. 31, 2022

Related U.S. Application Data
(63) Continuation-in-part of application No. PCT/US2022/038941, filed on Jul. 29, 2022.
Application No. PCT/US2022/038941 is a continuation-in-part of application No. PCT/US2022/032250, filed on Jun. 3, 2022.
(60) Provisional application No. 63/250,912, filed on Sep. 30, 2021.
(60) Provisional application No. 63/238,851, filed on Aug. 31, 2021.

(51) Int. Cl. G16H 50/20; G06Q 40/08

(52) U.S. Cl. CPC G16H 50/20; G06Q 40/08

(57) ABSTRACT

A computer may access one or more medical data tables storing medical insurance transaction data for a plurality of patients. The one or more medical data tables comprise a date column and a diagnosis column. The computer may identify, based on the diagnosis column, a set of patients having a biological condition. The set of patients can be from among the plurality of patients. The computer may determine, for each patient in the set of patients, an earliest date when the patient received a diagnosis of the biological condition. The computer may identify, based on the diagnosis column and the date column, a cohort of patients from the set of patients. The cohort of patients can lack a diagnosis from a collection of biological conditions associated with a date occurring during a predefined time window before the earliest date when the patient received the diagnosis of the biological condition.

### PRIORITY CLAIM AND INCORPORATION BY REFERENCE

This application claims priority to U.S. Provisional Patent Application Ser. No. 63/238,851 filed Aug. 31, 2021, and entitled “Data Repository, System, and Method for Cohort Selection”, to U.S. Provisional Patent Application Ser. No. 63/250,912 filed Sep. 30, 2021, and entitled “Computer Architecture for Generating a Reference Data Table, is a Continuation-in-Parts to PCT Application No. PCT/US2022/032250 filed Jun. 3, 2022, and entitled “Computer Architecture for Generating an Integrated Data Repository,” and is a Continuation-in-Parts to PCT Application No. PCT/US2022/038941 filed Jul. 29, 2022, and entitled “Computer Architecture for Identifying Lines of Therapy,” the entire contents of which are each incorporated by reference herein in their entirety.

### TECHNICAL FIELD

Implementations pertain to computer architecture. Some implementations relate to the use of computer systems to monitoring treatment and progress of biological conditions, including medical conditions and diseases. Some implementations relate to a data repository, system, and method for cohort selection.

### BACKGROUND

Precision medicine is an emerging approach for disease treatment and prevention that takes into account individual variability in one or more of genes, environment, and lifestyle for each person. This approach might allow doctors and researchers to predict more accurately which treatment and prevention strategies for a particular disease will work in which groups of people. It is in contrast to a one-size-fits-all approach, in which disease treatment and prevention strategies are developed for the average person, with less consideration for the differences between individuals. For some biological conditions, such as cancer, different people receive very different treatments. Identifying cohorts of patients that have similar biological conditions (e.g., medical conditions, diseases, or genetic profiles) may be desirable to study treatment or progression of the biological conditions.

When patients receive medical care, the medical service providers generate medical records indicating the treatment received by the patients. In addition, the medical records may indicate one or more diagnoses that correspond to the patients. The information included in the medical records is typically complex and/or is difficult to analyze in such a way that insights may be determined regarding the medical treatment provided to the patients.

### BRIEF DESCRIPTION OF THE DRAWINGS

FIG. 1 illustrates an example system in which cohort selection may be implemented.

FIG. 2 illustrates an example of processing insurance claims data to extract information, in accordance with one or more implementations.

FIG. 3 illustrates an example of patient information that may be stored, in accordance with one or more implementations.

FIG. 4 is a flow chart of an example method for identifying primary lung cancer patients, in accordance with one or more implementations.

FIG. 5 is a flow chart of an example method for identifying primary lung cancer patients and corner cases, in accordance with one or more implementations.

FIG. 6 is a flow chart of an example method for identifying a last active date, in accordance with one or more implementations.

FIG. 7 is a flowchart of a first example process associated with assigning a patient to a cohort, in accordance with one or more implementations.

FIG. 8 is a flowchart of a second example process associated with assigning a patient to a cohort, in accordance with one or more implementations.

FIG. 9 is a flowchart of an example process associated with identifying a cohort of patients, in accordance with one or more implementations.

FIG. 10 illustrates an example medical data table, in accordance with one or more implementations.

FIG. 11 illustrates an example architecture to generate an integrated data repository that includes multiple types of healthcare data, in accordance with one or more implementations.

FIG. 12 illustrates an example framework corresponding to an arrangement of data tables in an integrated data repository, in accordance with one or more implementations.

FIG. 13 illustrates an architecture to generate one or more datasets from information retrieved from a data repository that integrates health related data from a number of sources, in accordance with one or more implementations.

FIG. 14 illustrates an architecture to generate an integrated data repository that includes de-identified health insurance claims data and de-identified genomics data, in accordance with one or more implementations.

FIG. 15 illustrates a framework to generate a dataset, by a data pipeline system, based on data stored by an integrated data repository, in accordance with some implementations.

FIG. 16 illustrates a system to determine cohorts of patients having at least a primary diagnosis of a biological condition, in accordance with one or more implementations.

FIG. 17 is a block diagram of a computing machine, in accordance with one or more implementations.

### DETAILED DESCRIPTION

The following description and the drawings sufficiently illustrate specific implementations to enable those skilled in the art to practice them. Other implementations may incorporate structural, logical, electrical, process, and other changes. Portions and features of some implementations may be included in, or substituted for, those of other implementations. Implementations set forth in the claims encompass all available equivalents of those claims.

As discussed above, identifying cohorts of patients that have similar biological conditions (e.g., medical conditions or diseases) may be desirable to study treatment or progression of the biological conditions. Some implementations are directed to identifying cohorts of patients based on medical data, pharmacy data, and/or insurance transaction data. In some implementations, a computer accesses one or more medical data tables storing medical insurance transaction data for a plurality of patients. The one or more medical data tables comprise a date column and a diagnosis column. The computer identifies, based on the diagnosis column, a set of patients having a specified biological condition (e.g., lung cancer). The set of patients is from among the plurality of patients. The computer determines, for each patient in the set of patients, an earliest date when the patient received a diagnosis of the specified biological condition (e.g., the earliest date when the patient was diagnosed with lung cancer). The computer also identifies, based on the diagnosis codes included in the medical data tables, a cohort of patients from among the set of patients. In one or more examples, the cohort of patients may be determined based on timing of when a diagnosis was recorded with respect to a timing that one or more additional biological conditions were diagnosed with respect to the patients. For example, in situations where multiple biological conditions were diagnosed for a patient within a window of time, the patient may be placed in a cohort that includes patients having multiple diagnoses. Additionally, in situations where a single diagnosis is identified for the patient within a predefined time window and the diagnosis is a first diagnosis for the patient, the computer may determine that the patient is included in a cohort that corresponds to a biological condition related to the single diagnosis. The computer provides an output representing the cohort.

Aspects of the present technology may be implemented as part of a computer system. The computer system may be one physical machine, or may be distributed among multiple physical machines, such as by role or function, or by process thread in the case of a cloud computing distributed model. In various implementations, aspects of the technology may be configured to run in virtual machines that in turn are executed on one or more physical machines. It will be understood by persons of skill in the art that features of the technology may be realized by a variety of different suitable machine implementations.

The system includes various engines, each of which is constructed, programmed, configured, or otherwise adapted, to carry out a function or set of functions. The term engine as used herein means a tangible device, component, or arrangement of components implemented using hardware, such as by an application specific integrated circuit (ASIC) or field-programmable gate array (FPGA), for example, or as a combination of hardware and software, such as by a processor-based computing platform and a set of program instructions that transform the computing platform into a special-purpose device to implement the particular functionality. An engine may also be implemented as a combination of the two, with certain functions facilitated by hardware alone, and other functions facilitated by a combination of hardware and software.

In an example, the software may reside in executable or non-executable form on a tangible machine-readable storage medium. Software residing in non-executable form may be compiled, translated, or otherwise converted to an executable form prior to, or during, runtime. In an example, the software, when executed by the underlying hardware of the engine, causes the hardware to perform the specified operations. Accordingly, an engine is physically constructed, or specifically configured (e.g., hardwired), or temporarily configured (e.g., programmed) to operate in a specified manner or to perform part or all of any operations described herein in connection with that engine.

Considering examples in which engines are temporarily configured, each of the engines may be instantiated at different moments in time. For example, where the engines comprise a general-purpose hardware processor core configured using software, the general-purpose hardware processor core may be configured as respective different engines at different times. Software may accordingly configure a hardware processor core, for example, to constitute a particular engine at one instance of time and to constitute a different engine at a different instance of time.

In certain implementations, at least a portion, and in some cases, all, of an engine may be executed on the processor(s) of one or more computers that execute an operating system, system programs, and application programs, while also implementing the engine using multitasking, multithreading, distributed (e.g., cluster, peer-peer, cloud, etc.) processing where appropriate, or other such techniques. Accordingly, each engine may be realized in a variety of suitable configurations, and should generally not be limited to any particular implementation exemplified herein, unless such limitations are expressly called out.

In addition, an engine may itself be composed of more than one sub-engines, each of which may be regarded as an engine in its own right. Moreover, in the implementations described herein, each of the various engines corresponds to a defined functionality; however, it should be understood that in other contemplated implementations, each functionality may be distributed to more than one engine. Likewise, in other contemplated implementations, multiple defined functionalities may be implemented by a single engine that performs those multiple functions, possibly alongside other functions, or distributed differently among a set of engines than specifically illustrated in the examples herein.

As used herein, the term “model” encompasses its plain and ordinary meaning. A model may include, among other things, one or more engines which receive an input and compute an output based on the input. The output may be a classification. For example, an image file may be classified as depicting a cat or not depicting a cat. Alternatively, the image file may be assigned a numeric score indicating a likelihood whether the image file depicts the cat, and image files with a score exceeding a threshold (e.g., 0.9 or 0.95) may be determined to depict the cat.

This document may reference a specific number of things (e.g., “six mobile devices”). Unless explicitly set forth otherwise, the numbers provided are examples only and may be replaced with any positive integer, integer or real number, as would make sense for a given situation. For example, “six mobile devices” may, in alternative implementations, include any positive integer number of mobile devices. Unless otherwise mentioned, an object referred to in singular form (e.g., “a computer” or “the computer”) may include one or multiple objects (e.g., “the computer” may refer to one or multiple computers).

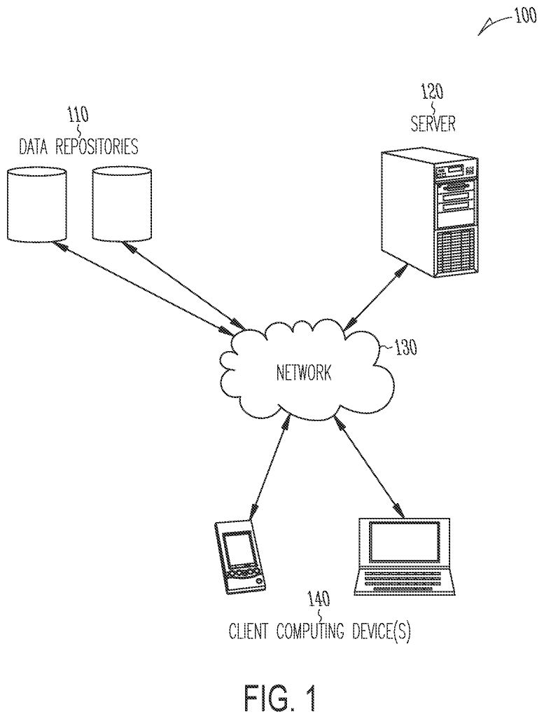

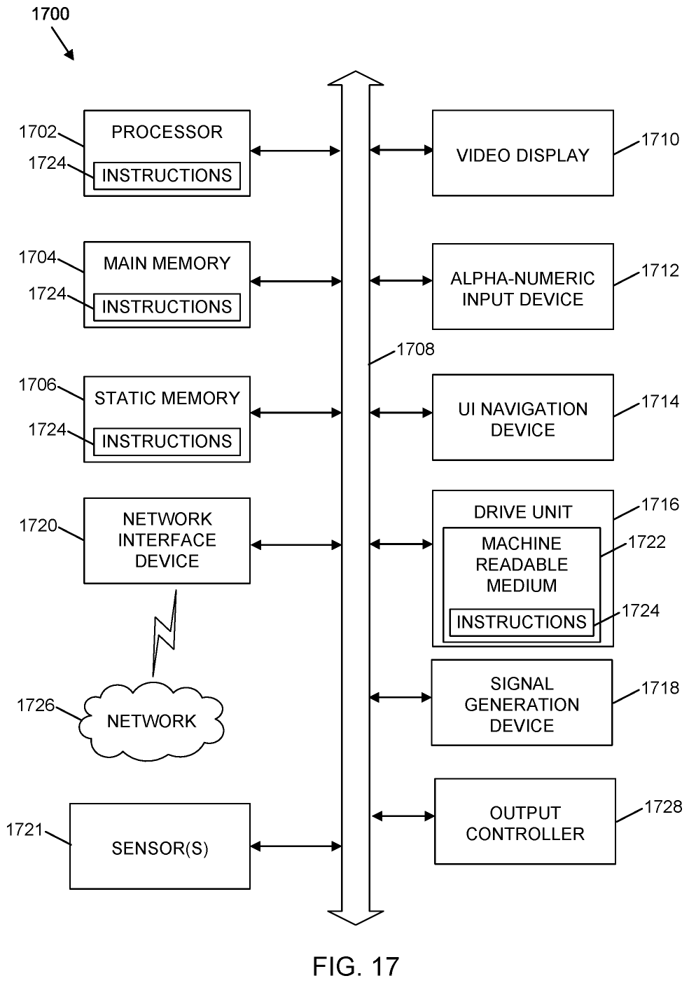

FIG. 1 illustrates an example system 100 in which cohort selection may be implemented. As shown, the system 100 includes data repositories 110, a server 120, and client computing device(s) 140 connected with one another via a network 130. The network 130 may include one or more of a wired network, a wireless network, a local area network, a wide area network, a virtual private network, the internet, an intranet, a Wi-Fi® network, a cellular network, and the like. Each of the data repositories 110, the server 120, and the client computing device(s) 140 may include all or a portion of the components of the computing machine 1700 shown in FIG. 17.

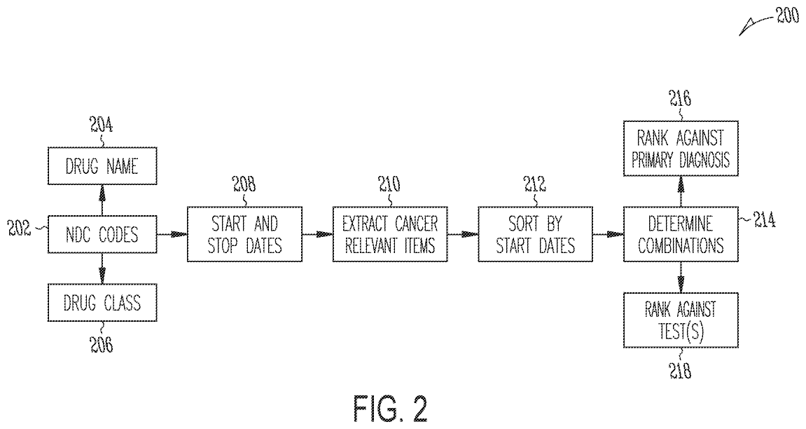

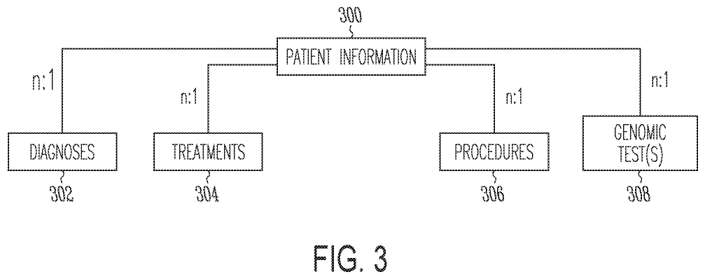

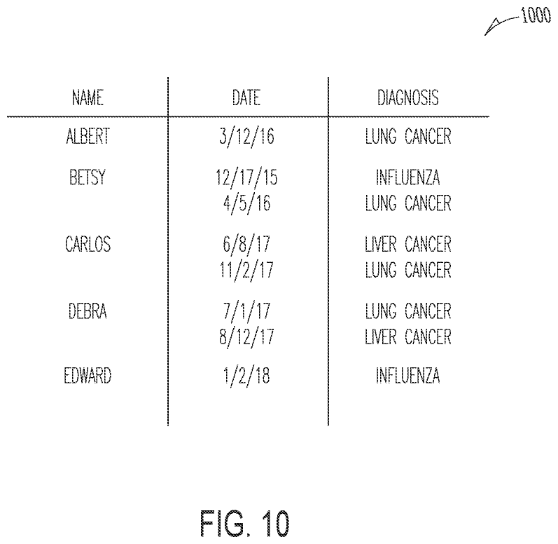

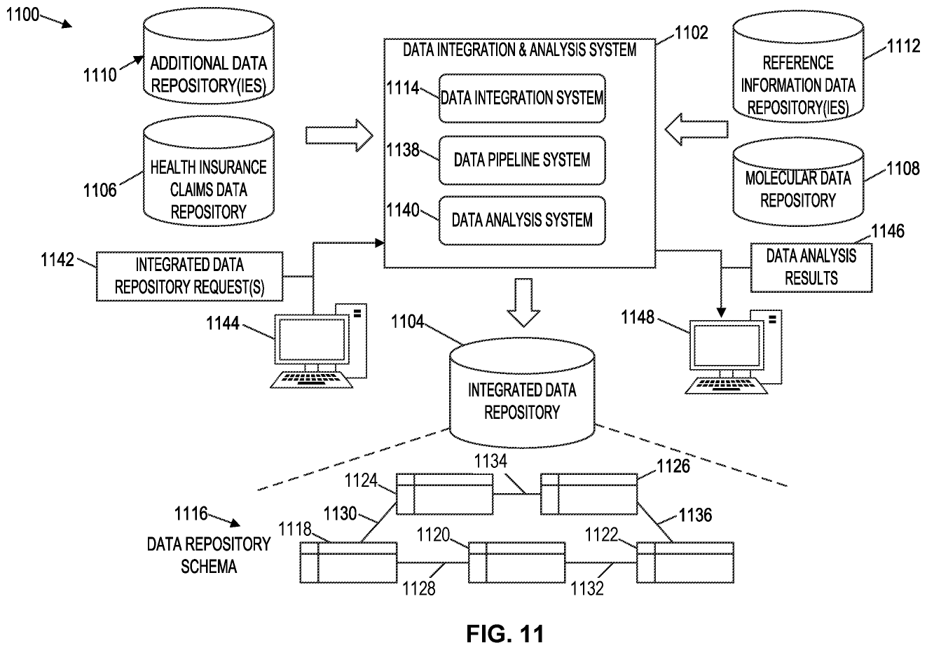

The data repositories 110 may be database(s) or other data storage unit(s). The data repositories 110 may store health insurance claims data, medical data, pharmacy data, genetic data, and the like. The data repositories 110 may include a single data repository or multiple data repositories. The data repositories 110 may store any of the data described herein, for example, the data shown in FIG. 2, FIG. 3, and FIG. 10. The data repositories 110 may include the data stored in the additional data repositories 1110, the health insurance claim data repository 1106, the reference information data repositories 1112, the molecular data repository 1108, and the integrated data repository 1104, shown in FIG. 11.

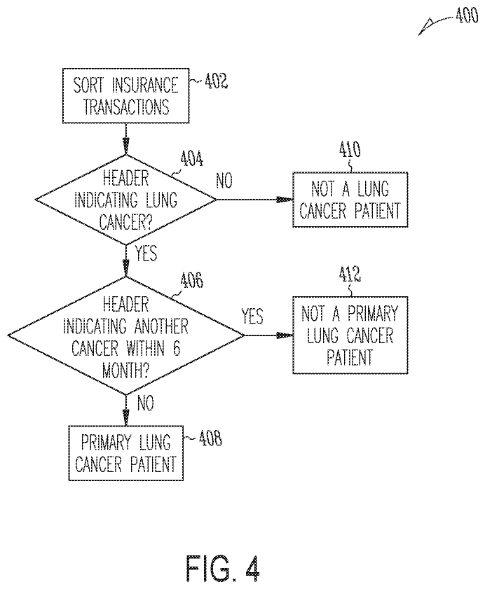

The client computing device(s) 140 may include one or more of a laptop computer, a desktop computer, a mobile phone, a tablet computer, a smart watch, a smart television including processing circuitry and a memory, and the like. The server 120 may include one or more servers arranged, for example, in a server farm. The server 120 may perform one or more of the processes described herein, for example, as shown in FIGS. 4-9.

As illustrated, the data repositories 110 and the server 120 are all connected to the network 130 and communicate with one another over the network 130. In alternative implementations, one or more of the data repositories 110 may be connected directly to the server 120 (e.g., using a direct wired or wireless connection), without going through the network 130. A data repository that is directly connected to the server 120 might or might not be connected to the network 130.

Precision medicine is playing an increasingly prominent role in the treatment of certain biological conditions. A wide array of patient subgroups with rare oncogenic driver mutations that are treatable with standard-of-care targeted therapies have now been identified.

In order to understand a patients' disease progression through treatments, it may be useful to consider the primary diagnoses, secondary/metastasis, molecular results, lines of treatment and the procedures performed on the patient.

Currently this information exists in the patient's insurance claims records as codes in medical headers, encounter summary and pharmacy records. The data is scattered across many columns and is difficult to query and derive the kind of information that can lend itself towards real world evidence. For example, codes need to go through multiple steps of transformation before they can be used meaningfully in queries.

FIG. 2 illustrates an example of processing 200 insurance claims data to extract information. The processing 200 begins at operation 202 with identifying National Drug Code (NDC) codes included in the insurance claims data. The NDC codes can indicate a drug used to treat a biological condition. The NDC codes can have one or more specified formats and the insurance claims data can be analyzed with respect to the one or more specified formats to identify NDC codes within the insurance claims data. Additionally, NDC codes can be located in one or more specified columns of the insurance claims data. In one or more examples, the one or more specified columns can be parsed and the rows in which values are present can be identified. The NDC codes can then be extracted from the insurance claims data. At operation 204, the NDC codes can be used to obtain drug name information and, at operation 206, the NDC codes can be used to obtain drug class information. The drug name information and the drug class information can be stored by a data repository that is accessible using one or more application programming interfaces.

At operation 208, the insurance claims data can be analyzed to determine start and stop dates for a given drug. At block 210, the insurance claims data can be analyzed to determine drugs provided to treat a patient that are relevant to a biological condition, such as cancer. In various examples, the NDC codes can be analyzed to identify drugs provided to a patient to treat the biological condition. At block 212, start dates are determined for the drugs that are provided to patients related to the given biological condition. At block 214, drug combinations are determined. For example, the insurance claims data can be analyzed to determine multiple drugs that may be provided to a patient to treat the biological condition. The combinations (of drugs and insurance claims) are ranked against a primary diagnosis at block 216 and ranked against test(s) (e.g., the Guardant 360 (G360) test by Guardant Health, Inc., of Redwood City, Calif.) at block 218.

As the dataset is built, (e.g., using processing 200) some implementations formalize the logic that is used in transforming: (i) NDC codes to lines of treatment, (ii) International Classification of Diseases (ICD) version 9 (ICD-9) and ICD-10 codes to primary diagnosis and metastases, and (iii) Healthcare Common Procedure Coding System (HCPCS), ICD-10, Personal Care Services (PCS), and Current Procedural Terminology (CPT) codes to procedures. The built dataset may include a subset of patients in a biological/medical data repository (e.g., database) that have a given biological condition (e.g., lung cancer) as a primary diagnosis in their insurance claims record. For this subset, some implementations sort patient information including diagnoses, treatments, procedures, and genomic tests.

FIG. 3 illustrates an example of patient information 300 that may be stored (e.g., in a data repository), in accordance with some implementations. As shown, the patient information 300 includes diagnoses 302, treatments 304, procedures 306, and genomic test(s) 308.

Some implementations attempt to build a framework for derived fields or derived content to evolve over time. Healthcare data may be messy and incomplete. Health care data may include diagnosis codes, treatment information, and dates. In spite of this, the ability to derive valuable contextual information and present the cancer journey of the patient with a high degree of confidence is the promise of real-world evidence. Some implementations describe what information to extract out of insurance claims data and how to transform them into meaningful higher order concepts that can be used to generate real world evidence, metrics and outcomes for the patients.

The dataset for a given biological condition, for example, lung cancer, is a subset of the patients in the overall dataset that have lung cancer as the primary diagnosis. Some implementations look into medical headers. Patients may have multiple medical header records, with each row representing one claim. Each claim may have multiple diagnosis codes. The diagnosis can be an ICD-9 or an ICD-10 code. A single claim may have either IDC-9 or ICD-10 code—there might be a mixture of both in the same claim. In some implementations, the column icd_type, or another code may be used to identify whether it is an ICD-9 or an ICD-10. The column claim_date will be used for the 6 month blackout period logic.

FIG. 4 is a flow chart of an example method 400 for identifying primary lung cancer patients (or patients with another primary diagnosis), in accordance with some implementations. The method 400 may be adjusted for other types of diagnoses that should not be coupled with other sets of diagnoses. For example, the method 400 may be used to identify patients diagnosed with pneumonia who have not been previously diagnosed with influenza.

At block 402, a computing machine (e.g., computing machine 1700) sorts the records associated with insurance transactions. In some implementations, the computing machine sorts the patient's medical headers in ascending order using the column for transaction date or claim date. One purpose of the sort is to identify the first occurrence of the lung cancer diagnosis, if one exists.

At block 404, the computing machine determines whether, for a given patient, a header indicates lung cancer (e.g., ICD code C34% or C33%) exists in the sorted insurance transactions. In some implementations, values in certain diagnosis code columns are analyzed to determine whether the C34 or C33 codes or the 162 code is present in these columns. The header indicating lung cancer may be identified based on ICD-9 or ICD-10 codes, for example, ICD code C34 for “malignant neoplasm of bronchus and lung,” ICD code C33 “malignant neoplasm of trachea” Or ICD coded 162 “Malignant neoplasm of trachea bronchus and lung.” If no such code exists, at block 410, the computing machine determines that the given patient is not a lung cancer patient. If such a header exists, the method 400 continues to block 406.

At block 406, the computing machine determines whether a header indicating another cancer different from lung cancer (e.g., breast cancer, prostate cancer, skin cancer, and the like) occurred within six months of a transaction date associated with the patient's earliest (in time) header indicating lung cancer. For example, the computing machine may look for any cancer-related ICD-10 or ICD-9 code within the diagnosis columns that occurred six months prior to the first lung-cancer related code identified at block 404. ICD-10 cancer codes may begin with C00 to C76. ICD-9 cancer codes may begin with or have the numbers 140-195. If such a header is identified, at block 412, the computing machine determines that this is not a primary lung cancer patient (e.g., another cancer metastasized to the patient's lungs). If no such code is identified, at block 408, the computing machine determines that this is a primary lung cancer patient. After block 408, block 410 or block 412, the method 400 ends.

Some implementations relate to the processing of corner cases. When there is a C34% code and another cancer code in the same earliest claim, some implementations may include this patient as a primary lung cancer patient. When there is a C34% code claim and another claim with the same claim date for the patient having non cancer code, some implementations may include the patient as a primary lung cancer patient. When there is 34% code claim which has another cancer claim within 6 months and then a claim for lung cancer after 9 months, some implementations might not include this patient as a primary lung cancer patient, since some implementations are looking for the first occurrence of a lung cancer diagnosis and applying a 6-month washout period before that date.

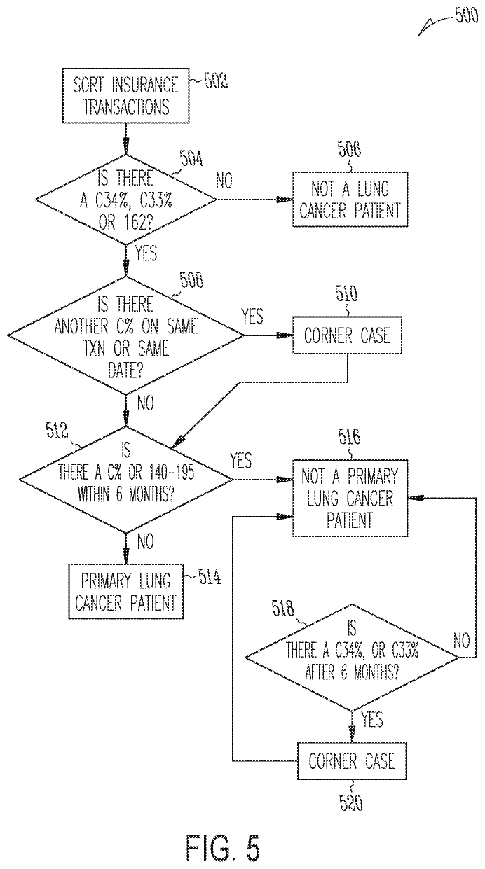

FIG. 5 is a flow chart of an example method 500 for identifying primary lung cancer patients and corner cases, in accordance with some implementations. Corner cases might be considered either as primary lung cancer patients or as non-primary lung cancer patients, depending on the needs of the user.

At block 502, a computing machine (e.g., computing machine 1700) sorts insurance transactions for a given patient.

At block 504, the computing machine determines whether there is a C34%, C33% or 162 ICD code. If not, at block 506, the computing machine determines that the given patient is not a lung cancer patient. After block 506, the method 500 ends. If so, the method 500 continues to block 508.

At block 508, the computing machine determines whether there is another C % on the same transaction or the same date. If so, the given patient is labeled as a corner case at block 510. After block 510, the method 500 continues to block 512. If not, the method 500 continues to block 512.

At block 512, the computing machine determines whether there is a C % or 140-195 ICD code within six months of the earliest transaction having a C34%, C33% or 162 ICD code identified at block 504. If not, at block 514, the given patient is labeled as primary lung cancer patient. After block 514, the method 500 ends. If so, at block 516, the patient is labeled as not a primary lung cancer patient.

At block 518, the computing machine determines whether there is another C34% or C33% after six or more months. If not, the patient remains labeled as not the primary lung cancer patient per block 516, and the method 500 ends. If so, the patient is labeled as a corner case at block 520, while also remaining labeled as not the primary lung cancer patient per block 516, and the method 500 ends. A corner case may be labeled as either a lung cancer patient or not a lung cancer patient depending on whether false positives (falsely identifying someone who is not a lung cancer patient as such) or false negatives (falsely identifying someone who is a lung cancer patient as not such) are preferred.

For deceased status of the patient, if the date of death for the patient exists, this value is set to yes. Otherwise, it is set to unknown. (In some cases, the deceased status for a patient may never be set to no. Alternatively, the deceased state may be set to no in the event that there is a confirmation, within a threshold time period (e.g., one day or seven days) before a current date, that the patient is alive.) In both cases, some implementations set a quality metric related to the deceased status to “high.” In one or more examples, the source of death data can include insurance claims information.

For patients that do not have a deceased status, the last active date column stores the latest date from patient headers and pharmacy claims in which a claim was generated. Some implementations include pharmacy claims which are designated as paid, pended or adjusted. To determine the last active date using a patient headers data table, the admission date column can be queried if it is present. In situations where a value is not present in the admission date column, the claim date column can be queried. To determine a last active date using a pharmacy claims data table, a date of service column can be queried.

The column including a value for age represents the age of the patient calculated from year of birth. If the death date is available, the value for age at death is the age of the patient at death.

The value for a patient for patient is metastatic status is either yes, no or unknown depending on the metastatic state of the patient and whether it is known. In a first case, if the claims records have a secondary malignancy code reported the patient is considered metastatic with high confidence. Secondary malignancy is identified by ICD-10 codes C77-C80 or ICD-9 196-198% seen after or on the same date/same claim as the primary diagnosis. When some implementations set patient metastatic status as “yes” using the logic above, some implementations may set the metastatic quality metric as “high.” In a second case, i

If the claims record has any cancer ICD-10/ICD-9 code different from the primary diagnosis code of the patient within 2 years (except skin and lung), some implementations may set patient metastatic status as “yes” and set the metastatic quality metric as “low.” Some ICD codes may be excluded. Lung Cancer ICD-9 codes to be excluded include: 162 Malignant neoplasm of trachea bronchus and lung. Lung Cancer ICD-10 codes to be excluded include: C34 Malignant neoplasm of bronchus and lung or C33 Malignant neoplasm of trachea. Skin Cancer ICD-9 codes to be excluded include: 172: Malignant melanoma of skin or 173 Other and unspecified neoplasm of skin. Skin Cancer ICD-10 codes to be excluded include: C43 Malignant melanoma of skin or C44 Other and unspecified neoplasm of skin.

Some implementations may set the patient metastatic status to unknown and also set the metastatic quality metric to “\n” since it is unknown.

The column indicating whether a patient is enrolled in a clinical trial ct is set to true if there is a Z006 ICD-10 or V707 ICD-9 code, and set to false otherwise. The column indicating the latest claim date related to a clinical trial corresponds to the latest transaction date when there is a Z006 ICD-10 or V707 ICD-9 code.

Data completeness may be based on percent of patients with no date of death and percent of patients with a high confidence of metastasis information. Data accuracy may be based on a count of patients with no date of death who are suspected to be deceased. Demographic data quality may be based on a percentage of patients with high quality demographic data.

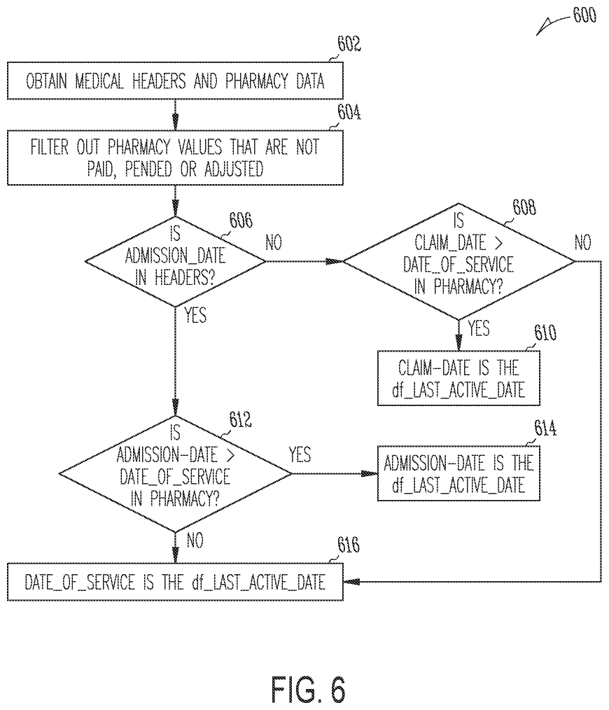

FIG. 6 is a flow chart of an example method 600 for identifying a last active date to be included in the patient information data table, in accordance with some implementations.

At block 602, a computing machine (e.g., computing machine 1700) obtains a dataset of medical headers and pharmacy data.

At block 604, the computing machine filters out pharmacy values that are not paid, pended or adjusted. Pharmacy values that are paid, pended or adjusted remain in the dataset.

At block 606, the computing machine determines whether an admission date is in the headers. If so, the method 600 continues to block 608. If not, the method 600 continues to block 612.

At block 608, the computing machine determines whether the header claim date is greater than the date of service in a pharmacy table. If so, the method 600 continues to block 610. If not, the method 600 continues to block 616.

At block 610, the computing machine determines that the claim date is the last active date. After block 610, the method 600 ends.

At block 612, the computing machine determines whether the admission date is subsequent to the date of service in pharmacy table. If so, the method 600 continues to block 614. If not, the method 600 continues to block 616.

At block 614, the computing machine determines that the admission date is the last active date. After block 614, the method 600 ends.

At block 616, the computing machine determines that the date of service is the last active date. After block 616, the method 600 ends.

In some implementations, a data table that includes patient information can be generated and the data stored by the data table can be used to determine one or more cohorts for a patient. One or more of the following patient information columns are included in the data table: year of birth, gender, date of death, deceased status, source of death data, last active date, patient metastatic status, clinical research study enrollment status, latest service date, remission status, age, and/or age at death.

It should be noted that some implementations determine primary diagnosis based on treatments (e.g., if a patient takes a drug that is used for lung cancer and not for other conditions, that patient likely has lung cancer). Some implementations may include a single table that establishes the relation between disease progression and treatment. The table may be generated based on common query patterns.

Some implementations relate to treatment data columns. The computing machine may create a Lung Cancer Pharmacy Table which contains cancer relevant treatments from Claims Pharmacy records for the lung cancer patients.

One of the main sources of information is the pharmacy records. The oral drugs are typically present in the pharmacy records. A small number of intravenous (IV) drugs can be present in the pharmacy records. Some implementations include treatments from the pharmacy records whose NDC codes have the class antineoplastics and transaction type paid or select cancer drug names listed in Table 2. The list in Table 2 is not exhaustive. It may be lacking some known, previously-existing cancer drugs and/or cancer drugs that are developed or identified in the future. Table 3 illustrates an example query. One purpose of the query of Table 3 is to extract, from the NDC reference table (or some other data repository), all cancer-relevant drugs. The query may be based on drug names, drug class, and the like. The query of Table 3 may be based on the example cancer drugs shown in Table 2.

TABLE 2
Example cancer drugs

Apalutamide
rucaparib
tucatinib
larotrectinib
trifluridine
entrectinib
niraparib
degarelix
abiraterone
dacomitinib
binimetinib
tofacitinib
tinidazole
encorafenib
topotecan
irinotecan
enzalutamide

TABLE 3
Example query

where ( (lower(nd.rxclassminconceptclassname) like ‘%antineo%’) or
(lower(nd.minconceptname) like ‘%antineo%’) or (lower(nd.minconceptname) like
‘%apalutamide%’) or (lower(nd.minconceptname) like ‘%rucaparib%’) or
(lower(nd.minconceptname) like ‘%tucatinib%’) or (lower(nd.minconceptname) like
‘%larotrectinib%’) or (lower(nd.minconceptname) like ‘%trifluridine%’) or
(lower(nd.minconceptname) like ‘%entrectinib%’) or (lower(nd.minconceptname) like
‘%niraparib%’) or (lower(nd.minconceptname) like ‘%degarelix%’) or
(lower(nd.minconceptname) like ‘%abiraterone%’) or (lower(nd.minconceptname) like
‘%dacomitinib%’) or (lower(nd.minconceptname) like ‘%binimetinib%’) or
(lower(nd.minconceptname) like ‘%tofacitinib%’) or (lower(nd.minconceptname) like
‘%tinidazole%’) or (lower(nd.minconceptname) like ‘%encorafenib%’) or
(lower(nd.minconceptname) like ‘%topotecan%’) or (lower(nd.minconceptname) like
‘%irinotecan%’) or (lower(nd.minconceptname) like ‘%enzalutamide%’) ) and
upper(ph.transaction_type) IN (‘PAID’)

Examples of pharmacy data columns in the pharmacy data table include: patient identifier (ID), date prescription written, quantity prescribed, number of refills authorized, pharmacy data of service, NDC code, pharmacy start date, pharmacy end date, drug name, drug class, drug category, fill number, days' supply, quantity dispensed, units of measure. Some implementations remove pharmacy claim duplicates. Duplicate pharmacy transactions may be defined as transactions with the same drug name and same days of supply on the same date of service.

Some implementations aggregate pharmacy claim days of supply on same days. Some implementations aggregate all the days of supply for drugs that appear multiple times on the same day with different days of supply. Table 4 illustrates an example of pharmacy data that may be stored. Cancer-relevant treatments may be extracted from service lines (e.g., for storage in a data structure such as Table 4).

TABLE 4
Example of stored data

| | | | Pharmacy | | | | | | Calculated |
|---|---|---|---|---|---|---|---|---|---|
| Patient | Date Rx | # | date of | | Fill | Days' | Quantity | Pharmacy | pharmacy |
| id | written | refills | service | Drug Name | # | supply | dispensed | start date | end date |
| 1 | 2016 Oct. 20 | 1 | 2016 Oct. 27 | osimertinib | 0 | 30 | 30 | 2016 Oct. 27 | 2016 Nov. 26 |
| 2 | 2016 Oct. 20 | 1 | 2016 Dec. 6 | dexamethasone | 1 | 30 | 30 | 2016 Dec. 6 | 2017 Jan. 5 |
| 3 | 2017 Jan. 10 | 1 | 2017 Jan. 11 | osimertinib | 0 | 30 | 30 | 2017 Jan. 11 | 2017 Feb. 10 |
| 4 | 2017 Jan. 10 | 1 | 2017 Jan. 12 | dexamethasone | 0 | 26 | 26 | 2017 Jan. 12 | 2017 Feb. 7 |
| 5 | 2017 Jan. 10 | 1 | 2017 Jan. 12 | osimertinib | 0 | 23 | 23 | 2017 Jan. 12 | 2017 Feb. 4 |
| 6 | 2017 Jan. 10 | 1 | 2017 Jan. 12 | dexamethasone | 0 | 16 | 16 | 2017 Jan. 12 | 2017 Jan. 28 |
| 7 | 2017 Jan. 10 | 1 | 2017 Jan. 12 | osimertinib | 0 | 14 | 14 | 2017 Jan. 12 | 2017 Jan. 26 |
| 8 | 2017 Jan. 10 | 1 | 2017 Jan. 12 | dexamethasone | 0 | 30 | 30 | 2017 Jan. 12 | 2017 Feb. 11 |

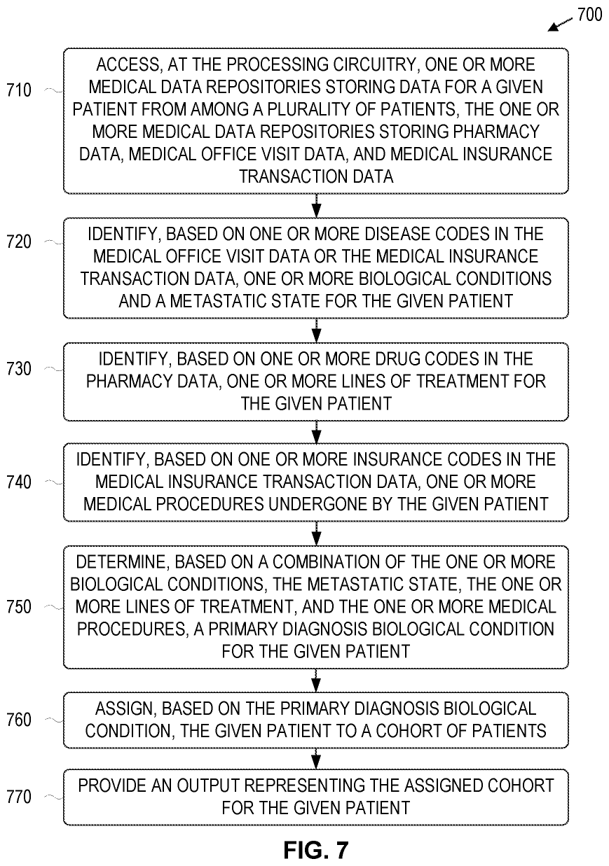

FIG. 7 is a flowchart of an example process 700 associated with assigning a patient to a cohort. In some implementations, one or more process blocks of FIG. 7 may be performed by a computing machine (e.g., computing machine 1700). In some implementations, one or more process blocks of FIG. 7 may be performed by another device or a group of devices separate from or including the computing machine. Additionally, or alternatively, one or more process blocks of FIG. 7 may be performed by one or more components of the computing machine 1700 shown in FIG. 17.

As shown in FIG. 7, process 700 may include accessing, by the computing machine and at the processing circuitry, one or more medical data repositories storing data for a given patient from among a plurality of patients. The one or more medical data repositories storing pharmacy data, medical office visit data, and medical insurance transaction data (block 710).

As further shown in FIG. 7, process 700 may include identifying, by the computing machine and based on one or more disease codes in the medical office visit data or the medical insurance transaction data, one or more biological conditions and a metastatic state for the given patient (block 720).

As further shown in FIG. 7, process 700 may include identifying, by the computing machine and based on one or more drug codes in the pharmacy data, one or more lines of treatment for the given patient (block 730).

As further shown in FIG. 7, process 700 may include identifying, by the computing machine and based on one or more insurance codes in the medical insurance transaction data, one or more medical procedures undergone by the given patient (block 740).

As further shown in FIG. 7, process 700 may include determining, by the computing machine and based on a combination of the one or more biological conditions, the metastatic state, the one or more lines of treatment, and the one or more medical procedures, a primary diagnosis biological condition for the given patient (block 750).

As further shown in FIG. 7, process 700 may include assigning, by the computing machine and based on the primary diagnosis biological condition, the given patient to a cohort of patients (block 760).

As further shown in FIG. 7, process 700 may include providing, by the computing machine, an output representing the assigned cohort for the given patient (block 770).

Process 700 may include additional implementations, such as any single implementation or any combination of implementations described below and/or in connection with one or more other processes described elsewhere herein.

In some implementations, determining the primary diagnosis biological condition comprises creating, based on the medical insurance transaction data, a master gap table indicating intervals between two successive procedures for the given patient that have a same treatment name and insurance code. The master gap table comprises columns for treatment name, insurance code, units, and gap length. In addition, based on the master gap table, a median gap table can be generated indicating the median gap for each combination of treatment name and insurance code. The median gap table comprises columns for treatment name, insurance code, units, and gap length. The determination of the primary diagnosis biological condition can be based, at least in part, on data in the median gap table. In some cases, the treatment that a patient is receiving can be an indicator of a biological condition that is present in the patient. For example, a patient who takes a known lung cancer drug and undergoes a known lung cancer therapy at a medical facility likely has lung cancer.

In some implementations, the processing circuitry comprises a plurality of multithreaded graphics processing units (GPUs), the method further comprising determining, in parallel and using parallel threads of the plurality of multithreaded GPUs, assigned cohorts for multiple patients, including the given patient, from the plurality of patients.

In some implementations, the disease codes comprise International Classification of Diseases (ICD) codes, the drug codes comprise National Drug Code (NDC) codes, and the insurance codes comprise Healthcare Common Procedure Coding System (HCPCS) codes.

In some implementations, process 700 includes identifying, based on the one or more disease codes in the medical office visit data or the medical insurance transaction data, the one or more biological conditions for the given patient comprise identifying that the given patient has lung cancer based on ICD codes associated with lung cancer.

In some implementations, process 700 includes identifying, based on the one or more disease codes in the medical office visit data or the medical insurance transaction data, the metastatic state for the given patient is based on a secondary malignancy ICD code or HCPCS code.

In some implementations, the primary diagnosis biological condition for the given patient is determined based on disease codes, drug codes or insurance codes associated with a date within a predefined date range.

Although FIG. 7 shows example blocks of process 700, in some implementations, process 700 may include additional blocks, fewer blocks, different blocks, or differently arranged blocks than those depicted in FIG. 7. Additionally, or alternatively, two or more of the blocks of process 700 may be performed in parallel.

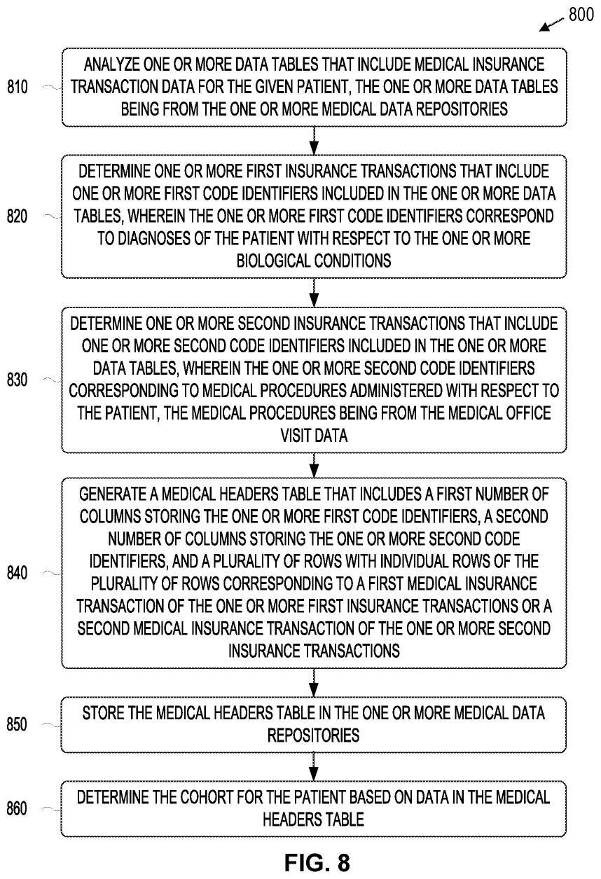

FIG. 8 is a flowchart of an example process 800 associated with assigning a given patient to a cohort. In some implementations, one or more process blocks of FIG. 8 may be performed by a computing machine (e.g., computing machine 1700). In some implementations, one or more process blocks of FIG. 8 may be performed by another device or a group of devices separate from or including the computing machine. Additionally, or alternatively, one or more process blocks of FIG. 8 may be performed by one or more components of the computing machine 1700 shown in FIG. 17.

As shown in FIG. 8, process 800 may include analyzing, by the computing machine, one or more data tables that include medical insurance transaction data for the given patient. The one or more data tables can be obtained from the one or more medical data repositories (block 810).

As further shown in FIG. 8, process 800 may include determining, by the computing machine, one or more first insurance transactions that include one or more first code identifiers included in the one or more data tables, wherein the one or more first code identifiers correspond to diagnoses of the patient with respect to the one or more biological conditions (block 820).

As further shown in FIG. 8, process 800 may include determining, by the computing machine, one or more second insurance transactions that include one or more second code identifiers included in the one or more data tables, wherein the one or more second code identifiers corresponding to medical procedures administered with respect to the patient, the medical procedures being from the medical office visit data (block 830).

As further shown in FIG. 8, process 800 may include generating, by the computing machine, a medical headers table that includes a first number of columns storing the one or more first code identifiers, a second number of columns storing the one or more second code identifiers, and a plurality of rows with individual rows of the plurality of rows corresponding to a first medical insurance transaction of the one or more first insurance transactions or a second medical insurance transaction of the one or more second insurance transactions (block 840).

As further shown in FIG. 8, process 800 may include storing, by the computing machine, the medical headers table in the one or more medical data repositories (block 850).

As further shown in FIG. 8, process 800 may include determining, by the computing machine, the cohort for the patient based on data in the medical headers table (block 860).

Process 800 may include additional implementations, such as any single implementation or any combination of implementations described below and/or in connection with one or more other processes described elsewhere herein.

In some implementations, the one or more first insurance transactions indicate a date of service of the individual first insurance transactions, and the one or more second insurance transactions indicate a date of service of the individual second insurance transactions.

In some implementations, process 800 includes arranging the plurality of rows of the medical headers table in ascending order based on the dates of service such that a claim with an earliest date of service is a first row of the medical headers table and that a transaction with a most recent date of service is a last row of the medical headers table.

In some implementations, process 800 may include analyzing the one or more first code identifiers to determine that a first code identifier of the one or more first code identifiers is included in a group of insurance code identifiers that correspond to the one or more biological conditions.

In some implementations, the first code identifier is arranged according to a first format that corresponds to a first classification of insurance code identifiers, the first classification of insurance code identifiers corresponds to International Classification of Diseases version 9 (ICD-9).

In some implementations, the first code identifier is arranged according to a second format that corresponds to a second classification of insurance code identifiers, the second classification of insurance code identifiers corresponds to International Classification of Diseases version 10 (ICD-10).

In some implementations, the one or more biological conditions include a plurality of subtypes, individual subtypes of the plurality of subtypes correspond to a subset of the group of insurance code identifiers that correspond to the one or more biological conditions, and the method further comprises determining that the first code identifier is included in a first subset of the group of insurance codes that corresponds to a first subtype of the biological condition.

In some implementations, the biological condition is cancer and the plurality of subtypes include at least one of lung cancer, breast cancer, or colorectal cancer.

In some implementations, process 800 includes determining one or more third insurance transactions having a date of service that is within a predefined time period ending on a first date of service, analyzing one or more third insurance code identifiers of the third insurance transactions with respect to the first code identifiers and the second code identifiers.

In some implementations, process 800 includes determining that the one or more third insurance code identifiers are not included in the first code identifiers and the second code identifiers, and determining, based on the third insurance code identifiers, that the patient is included in a cohort of patients in which a given subtype of the one or more biological conditions is present.

In some implementations, the one or more third insurance code identifiers correspond to an additional biological condition.

In some implementations, process 800 includes determining that the one or more third insurance code identifiers are included in a portion of the group of insurance code identifiers that are not included in the subset of the group of insurance code identifiers, determining that a date of service of at least one of the one or more third insurance transactions is a same date as the date of service as one of the one or more first insurance transactions, determining that there are no other additional insurance transactions having insurance code identifiers included in the group of code identifiers, and determining that the patient is included in a cohort of patients in which the subtype of the biological condition is present.

In some implementations, process 800 includes determining that the one or more third insurance code identifiers are included in a portion of the group of insurance code identifiers that are not included in the subset of the group of insurance code identifiers, determining that a date service of at least one of the one or more third insurance claim is prior to the date of service of one of the one or more first insurance transactions and within the predefined time period, and determining that the patient is not included in a cohort of patients in which the subtype of the biological condition is present.

Although FIG. 8 shows example blocks of process 800, in some implementations, process 800 may include additional blocks, fewer blocks, different blocks, or differently arranged blocks than those depicted in FIG. 8. Additionally, or alternatively, two or more of the blocks of process 800 may be performed in parallel.

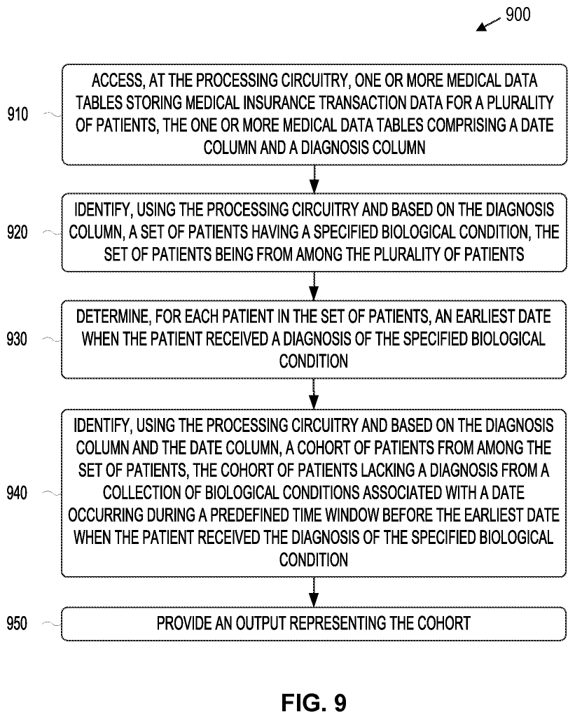

FIG. 9 is a flowchart of an example process 900 associated with identifying a cohort of patients. In some implementations, one or more process blocks of FIG. 9 may be performed by a computing machine (e.g., computing machine 1700). In some implementations, one or more process blocks of FIG. 9 may be performed by another device or a group of devices separate from or including the computing machine. Additionally, or alternatively, one or more process blocks of FIG. 9 may be performed by one or more components of the computing machine 1700 shown in FIG. 17.

As shown in FIG. 9, process 900 may include accessing, by the computing machine and at the processing circuitry, one or more medical data tables storing medical insurance transaction data for a plurality of patients. The one or more medical data tables comprise a date column and a diagnosis column (block 910).

As further shown in FIG. 9, process 900 may include identifying, by the computing machine and using the processing circuitry and based on the diagnosis column, a set of patients having a specified biological condition, the set of patients being from among the plurality of patients (block 920).

As further shown in FIG. 9, process 900 may include determining, by the computing machine and for each patient in the set of patients, an earliest date when the patient received a diagnosis of the specified biological condition (block 930).

As further shown in FIG. 9, process 900 may include identifying, by the computing machine, using the processing circuitry, and based on the diagnosis column and the date column, a cohort of patients from among the set of patients, the cohort of patients lacking a diagnosis from a collection of biological conditions associated with a date occurring during a predefined time window before the earliest date when the patient received the diagnosis of the specified biological condition (block 940).

As further shown in FIG. 9, process 900 may include providing, by the computing machine, an output representing the cohort (block 950).

Process 900 may include additional implementations, such as any single implementation or any combination of implementations described below and/or in connection with one or more other processes described elsewhere herein.

In some implementations, the diagnosis column stores International Classification of Diseases version 9 (ICD-9) or International Classification of Diseases version 10 (ICD-10) codes.

In some implementations, the specified biological condition is lung cancer, wherein the collection of biological conditions comprises cancers different from lung cancer, wherein the predefined time window before the earliest date is six months before the earliest date.

In some implementations, the specified biological condition is a specified type of cancer, the method further comprising determining a metastatic state of at least one patient from the cohort.

In some implementations, the metastatic state is determined based on a secondary malignancy International Classification of Diseases (ICD) code or Healthcare Common Procedure Coding System (HCPCS) code.

In some implementations, identifying the cohort comprises arranging rows associated with patients in the set by date, and accessing rows associated with the predefined time window to identify patients in the set that lack the diagnosis from the collection of biological conditions during the predefined time window.

Although FIG. 9 shows example blocks of process 900, in some implementations, process 900 may include additional blocks, fewer blocks, different blocks, or differently arranged blocks than those depicted in FIG. 9. Additionally, or alternatively, two or more of the blocks of process 900 may be performed in parallel.

FIG. 10 illustrates an example medical data table 1000, in accordance with some implementations. The table 1000 includes data (name, date, and diagnosis) about patients: Albert, Betsy, Carlos, Debra, and Edward, who are candidates for inclusion in a lung cancer primary diagnosis cohort. Some implementations may store a patient ID number instead of a name to ensure patient privacy. It should be noted that the table 1000 is simplified in order to illustrate how some implementations operate. Other tables used with the technology disclosed herein may include more rows, columns, patients, and data.

Albert's only diagnosis is lung cancer, so Albert is included in the lung cancer primary diagnosis cohort.

Betsy was diagnosed with influenza on Dec. 17, 2015 and with lung cancer on Apr. 5, 2016. While Betsy was diagnosed with influenza before being diagnosed with lung cancer, Betsy is still included in the lung cancer primary diagnosis cohort because influenza is not a type of cancer.

Carlos was diagnosed with liver cancer on Jun. 8, 2017 and with lung cancer on Nov. 2, 2017. Since Carlos' earliest lung cancer diagnosis has another cancer diagnosis (liver cancer) within six months before the lung cancer diagnosis, Carlos' primary cancer is liver cancer, not lung cancer. Since Carlos' primary cancer is not lung cancer, Carlos is not included in the lung cancer primary diagnosis cohort.

Debra was diagnosed with lung cancer on Jul. 1, 2017 and with liver cancer on Aug. 12, 2017. As lung cancer was the first cancer with which Debra was diagnosed, Debra is included in the lung cancer primary diagnosis cohort.

Edward was diagnosed with influenza on Jan. 2, 2018, but was never diagnosed with lung cancer or any other cancer. Thus, Edward is not included in the lung cancer primary diagnosis cohort. Based on the table 1000, the lung cancer primary diagnosis cohort includes Albert, Betsy, and Debra. The lung cancer primary diagnosis cohort does not include Carlos and Edward.

FIG. 11 illustrates an example architecture 1100 to generate an integrated data repository that includes multiple types of healthcare data, according to one or more implementations. The architecture 1100 may include a data integration and analysis system 1102. The data integration and analysis system 1102 may obtain data from a number of data sources and integrate the data from the data sources into an integrated data repository 1104. For example, the data integration and analysis system 1102 may obtain data from a health insurance claims data repository 1106. In various examples, the data integration and analysis system 1102 and the health insurance claims data repository 1106 may be created and maintained by different entities. In one or more additional examples, the data integration and analysis system 1102 and the health insurance claims data repository 1106 may be created and maintained by a same entity.

The data integration and analysis system 1102 may be implemented by one or more computing devices. The one or more computing devices may include one or more server computing devices, one or more desktop computing devices, one or more laptop computing devices, one or more tablet computing devices, one or more mobile computing devices, or combinations thereof. In certain implementations, at least a portion of the one or more computing devices may be implemented in a distributed computing environment. For example, at least a portion of the one or more computing devices may be implemented in a cloud computing architecture. In scenarios where the computing systems used to implement the data integration and analysis system 1102 are configured in a distributed computing architecture, processing operations may be performed concurrently by multiple virtual machines. In various examples, the data integration and analysis system 1102 may implement multithreading techniques. The implementation of a distributed computing architecture and multithreading techniques cause the data integration and analysis system 1102 to utilize fewer computing resources in relation to computing architectures that do not implement these techniques.

The health insurance claims data repository 1106 may store information obtained from one or more health insurance companies that corresponds to insurance claims made by subscribers of the one or more health insurance companies. The health insurance claims data repository 1106 may be arranged (e.g., sorted) by patient identifier. The patient identifier may be based on the patient's first name, last name, date of birth, social security number, address, employer, and the like. The data stored by the health insurance claims data repository 1106 may include structured data that is arranged in one or more data tables. The one or more data tables storing the structured data may include a number of rows and a number of columns that indicate information about health insurance claims made by subscribers of one or more health insurance companies in relation to procedures and/or treatments received by the subscribers from healthcare providers. At least a portion of the rows and columns of the data tables stored by the health insurance claims data repository 1106 may include health insurance codes that may indicate diagnoses of biological conditions, and treatments and/or procedures obtained by subscribers of the one or more health insurance companies. In various examples, the health insurance codes may also indicate diagnostic procedures obtained by individuals that are related to one or more biological conditions that may be present in the individuals. In one or more examples, a diagnostic procedure may provide information used in the detection of the presence of a biological condition. A diagnostic procedure may also provide information used to determine a progression of a biological condition. In one or more illustrative examples, a diagnostic procedure may include one or more imaging procedures, one or more assays, one or more laboratory procedures, one or more combinations thereof, and the like.

The data integration and analysis system 1102 may also obtain information from a molecular data repository 1108. The molecular data repository 1108 may store data of a number of individuals related to genomic information, genetic information, pathology information (e.g., analysis of tissue slides), metabolomic information, transcriptomic information, fragmentomic information, immune receptor information, methylation information, epigenomic information, and/or proteomic information. In one or more examples, the data integration and analysis system 1102 and the molecular data repository 1108 may be created and maintained by different entities. In one or more additional examples, the data integration and analysis system 1102 and the molecular data repository 1108 may be created and maintained by a same entity.

The genomic information may indicate one or more mutations corresponding to genes of the individuals. A mutation to a gene of individuals may correspond to differences between a sequence of nucleic acids of the individuals and one or more reference genomes. The reference genome may include a known reference genome, such as hg19. In various examples, a mutation of a gene of an individual may correspond to a difference in a germline gene of an individual in relation to the reference genome. In one or more additional examples, the reference genome may include a germline genome of an individual. In one or more further examples, a mutation to a gene of an individual may include a somatic mutation. Mutations to genes of individuals may be related to insertions, deletions, single nucleotide variants, loss of heterozygosity, duplication, amplification, translocation, fusion genes, or one or more combinations thereof.

In one or more illustrative examples, genomic information stored by the molecular data repository 1108 may include genomic profiles of tumor cells present within individuals. In these situations, the genomic information may be derived from an analysis of genetic material, such as deoxyribonucleic acid (DNA) and/or ribonucleic acid (RNA) from a sample, including, but not limited to, a tissue sample or tumor biopsy, circulating tumor cells (CTCs), exosomes or efferosomes, or from circulating nucleic acids (e.g., cell-free DNA) found in blood samples of individuals that is present due to the degradation of tumor cells present in the individuals. In one or more examples, the genomic information of tumor cells of individuals may correspond to one or more target regions. One or more mutations present with respect to the one or more target regions may indicate the presence of tumor cells in individuals. The genomic information stored by the molecular data repository 1108 may be generated in relation to an assay or other diagnostic test that may determine one or more mutations with respect to one or more target regions of the reference genome.

“Cell-free DNA,” “cfDNA molecules,” or simply “cfDNA” include DNA molecules that occur in a subject in extracellular form (e.g., in blood, serum, plasma, or other bodily fluids such as lymph, cerebrospinal fluid, urine, or sputum) and includes DNA not contained within or otherwise bound to a cell at the point of isolation from the subject. While the DNA originally existed in a cell or cells of a large complex biological organism (e.g., a mammal) or other cells, such as bacteria, colonizing the organism, the DNA has undergone release from the cell(s) into a fluid found in the organism. cfDNA includes, but is not limited to, cell-free genomic DNA of the subject (e.g., a human subject's genomic DNA) and cell-free DNA of microbes, such as bacteria, inhabiting the subject (whether pathogenic bacteria or bacteria normally found in commonly colonized locations such as the gut or skin of healthy controls), but does not include the cell-free DNA of microbes that have merely contaminated a sample of bodily fluid. Typically, cfDNA may be obtained by obtaining a sample of the fluid without the need to perform an in vitro cell lysis step and also includes removal of cells present in the fluid (e.g., centrifugation of blood to remove cells).

In one or more additional examples, the data integration and analysis system 1102 may obtain information from one or more additional data repositories 1110. The one or more additional data repositories 1110 may store data related to electronic medical records of individuals for which data is present in at least one of the health insurance claims data repository 1106 or the molecular data repository 1108. Further, the one or more additional data repositories 1110 may store data related to pathology reports of individuals for which data is present in at least one of the health insurance claims data repository 1106 or the molecular data repository 1108. In various examples, the one or more additional data repositories 1110 may store data related to biological conditions and/or treatments for biological conditions. In one or more examples, the data integration and analysis system 1102 and at least a portion of the one or more additional data repositories 1110 may be created and maintained by different entities. In one or more further examples, the data integration and analysis system 1102 and at least a portion of the one or more additional data repositories 1110 may be created and maintained by a same entity.

In one or more further implementations, the data integration and analysis system 1102 may obtain information from one or more reference information data repositories 1112. The one or more reference information data repositories 1112 may store information that includes definitions, standards, protocols, vocabularies, one or more combinations thereof, and the like. In various examples, the information stored by the one or more reference information data repositories may correspond to biological conditions and/or treatments for biological conditions. In one or more illustrative examples, the one or more reference information data repositories 1112 may include RxNorm. (RxNorm provides normalized names for clinical drugs and links its names to many of the drug vocabularies used in pharmacy management and drug interaction software.) In one or more examples, the data integration and analysis system 1102 and at least a portion of the one or more reference information data repositories 1112 may be created and maintained by different entities. In one or more further examples, the data integration and analysis system 1102 and at least a portion of the one or more reference information data repositories 1112 may be created and maintained by a same entity.

The data integration and analysis system 1102 may obtain data from at least one of the health insurance claims data repository 1106, the molecular data repository 1108, the one or more additional data repositories 1110, or the reference information data repositories 1112 via one or more communication networks accessible to the data integration and analysis system 1102 and accessible to at least one of the health insurance claims data repository 1106, the molecular data repository 1108, the one or more additional data repositories 1110, or the reference information data repositories 1112. The data integration and analysis system 1102 may also obtain data from at least one of the health insurance claims data repository 1106, the molecular data repository 1108, the one or more additional data repositories 1110, or the reference information data repositories 1112 via one or more secure communication channels. In addition, the data integration and analysis system 1102 may obtain data from at least one of the health insurance claims data repository 1106, the molecular data repository 1108, the one or more additional data repositories 1110, or the reference information data repositories 1112 via one or more calls of an application programming interface (API).

The data integration and analysis system 1102 may include a data integration system 1114. The data integration system 1114 may obtain data from the health insurance claims data repository 1106 and the molecular data repository 1108 to generate the integrated data repository 1104. The data integration system 1114 may also obtain data from the one or more additional data repositories 1110 to generate the integrated data repository 1104. In various examples, the data integration system 1114 may implement one or more natural language processing techniques to integrate data from the one or more additional data repositories 1110 into the integrated data repository 1104.

In one or more examples, the data integration system 1114 may generate one or more tokens to identify individuals that have data stored in the health insurance claims data repository 1106 and that have data stored in the molecular data repository 1108. In various examples, the data integration system 1114 may generate one or more tokens by implementing one or more hash functions. The data integration system 1114 may implement the one or more hash functions to generate the one or more tokens based on information stored by at least one of the health insurance claims data repository 1106 or the molecular data repository 1108. For example, the information used by the data integration system 1114 to generate individual tokens by implementing a hash function may include at least one of an identifier of respective individuals, date of birth of the respective individuals, a postal code of the respective individuals, date of birth of the respective individuals, or a gender of the respective individuals. In one or more illustrative examples, the identifiers of the respective individuals may include a combination of at least a portion of a first name of the respective individuals and at least a portion of the last name of the respective individuals. Tokens generated using data from different data repositories may correspond to the same or similar information or the same or similar type stored by the different data repositories. To illustrate, tokens may be generated using a portion of names of individuals, date of birth, at least a portion of a postal code, and gender obtained from the health insurance claims data repository 1106 and the molecular data repository 1108.

The data integration system 1114 may integrate data from a number of different data sources by analyzing tokens generated by implementing one or more hash functions using data obtained from the number of different data sources. For example, the data integration system 1114 may obtain one or more first tokens generated from data stored by the health insurance claims data repository 1106 and one or more second tokens generated from data stored by the molecular data repository 1108. The data integration system 1114 may analyze the one or more first tokens with respect to the one or more second tokens to determine individual first tokens that correspond to individual second tokens. In one or more illustrative examples, the data integration system 1114 may identify individual first tokens that match individual second tokens. A first token may match a second token when the data of the first token has at least a threshold amount of similarity with respect to the data of the second token. In one or more examples, a first token may match a second token when the data of the first token is the same as the data of the second token. To illustrate, a first token may match a second token when an alphanumeric string of the first token is the same as an alphanumeric string of the second token.

By determining a first token generated using data stored by the health insurance claims data repository 1106 that corresponds to a second token generated using data stored by the molecular data repository 1108, the data integration system 1114 may identify an individual having data that is stored in both the health insurance claims data repository 1106 and in the molecular data repository 1108. In this way, the data integration system 1114 may obtain data from the health insurance claims data repository 1106 from a number of individuals and data from the molecular data repository 1108 from the same number of individuals and store the health insurance claims data and the molecular data for the number of individuals in the integrated data repository 1104.

The data integration system 1114 may also integrate data stored by the one or more additional data repositories 1110 with data from the health insurance claims data repository 1106 and the molecular data repository 1108 to generate the integrated data repository 1104. To illustrate, the data integration system 1114 may obtain one or more third tokens generated from data stored by an additional data repository 1110, such as a data repository storing data corresponding to pathology reports. The data integration system 1114 may analyze the one or more third tokens with respect to the first tokens generated using information stored by the health insurance claims data repository 1106 and the second tokens generated using information stored by the molecular data repository 1108 to determine respective third tokens that correspond to individuals first tokens and individual second tokens. In one or more illustrative examples, the data integration system 1114 may identify third tokens generated using one or more hash functions and a common set of information obtained from the health insurance claims data repository 1106, the molecular data repository 1108, and the additional data repository 1110.

By determining a third token generated using data stored by an additional data repository 1110 that corresponds to a first token generated using data stored by the health insurance claims data repository 1106 and a second token generated using data stored by the molecular data repository 1108, the data integration system 1114 may identify an individual having data that is stored in the health insurance claims data repository 1106, the molecular data repository 1108, and in an additional data repository 1110. In this way, the data integration system 1114 may obtain data from the health insurance claims data repository 1106 from a number of individuals and data from the molecular data repository 1108 and an additional data repository 1110 from the same number of individuals and store the health insurance claims data, the molecular data, and the additional data for the number of individuals in the integrated data repository 1104.

The data stored by the integrated data repository 1104 for the number of individuals may be accessible using respective identifiers of individuals. The data integration system 1114 may implement a number of techniques as part of a de-identification process with respect to storing and retrieving information of individuals in the integrated data repository 1104. The identifiers of individuals may correspond to keys that are generated using at least one hash function. The identifiers of the individuals may also be generated by implementing one or more salting processes with respect to the keys generated using the at least one hash function. the tokens generated using one or more hash functions and a common set of information obtained from the health insurance claims data repository 1106, the molecular data repository 1108, and/or the additional data repository 1110. In one or more illustrative examples, the identifiers generated by the data integration system 1114 to access information for respective individuals that is stored by the integrated data repository 1104 may be unique for each individual. In one or more examples, the identifiers of the individuals may be generated using at least a portion of the information used to generate the tokens related to the individuals. In one or more additional examples, the identifiers of the individuals may be generated using different information from the information used to generate the tokens related to the individuals.

The data integration system 1114 may also generate the integrated data repository 1104 from a number of different combinations of data repositories in a similar manner. For example, the data integration system 1114 may obtain tokens generated from information stored by the health insurance claims data repository 1106 and additional tokens generated from information stored by one or more additional data stores 1110. The data integration system 1114 may determine individual tokens generated from information stored by the health insurance claims data repository 1106 that correspond to individual additional tokens generated from information stored by the one or more additional data repositories 1110. By determining tokens generated using data stored by the health insurance claims data repository 1106 that correspond to additional tokens generated using data stored by an additional data repository 1110, the data integration system 1114 may identify individuals having data that is stored in both the health insurance claims data repository 1106 and in the additional data repository 1110. In this way, the data integration system 1114 may obtain data from the health insurance claims data repository 1106 from a number of individuals and data from the additional data repository 1110 from the same number of individuals and store the health insurance claims data and the additional data for the number of individuals in the integrated data repository 1104. The health insurance claims data and the additional data stored by the integrated data repository 1104 for the number of individuals may be accessible using respective identifiers of individuals.

In one or more further examples, the data integration system 1114 may obtain tokens generated from information stored by the molecular data repository 1108 and tokens generated from information stored by one or more additional data stores 1110. The data integration system 1114 may determine individual tokens generated from information stored by the molecular data repository 1108 that correspond to individual additional tokens generated from information stored by the one or more additional data repositories 1110. By determining tokens generated using data stored by the molecular data repository 1108 that correspond to additional tokens generated using data stored by an additional data repository 1110, the data integration system 1114 may identify individuals having data that is stored in both the molecular data repository 1108 and in the additional data repository 1110. In this way, the data integration system 1114 may obtain data from the molecular data repository 1108 from a number of individuals and data from the additional data repository 1110 from the same number of individuals and store the molecular data and the additional data for the number of individuals in the integrated data repository 1104. The molecular data and the additional data stored by the integrated data repository 1104 for the number of individuals may be accessible using respective identifiers of individuals.

The data stored by the integrated data repository 1104 may be stored according to one or more regulatory frameworks that protect the privacy and ensure the security of medical records, health information, and insurance information of individuals. For example, data may be stored by the integrated data repository 1104 in accordance with one or more governmental regulatory frameworks directed to protecting personal information, such as the Health Insurance Portability and Accountability Act (HIPAA) and/or the General Data Protection Regulation (GDPR). The integrated data repository 1104 also stores data in an anonymized and de-identified manner to ensure protection of the privacy of individuals that have data stored by the integrated data repository 1104. To further ensure the privacy of individuals that have data stored by the integrated data repository 1104, the data integration system 1114 may re-generate the integrated data repository 1104 periodically. For example, the data integration system 1114 may create the integrated data repository 1104 once per quarter. In one or more additional examples, the data integration system 1114 may generated the integrated data repository 1104 on a monthly basis, on a weekly basis, or once every two weeks. By re-generating the integrated data repository 1104 on a periodic basis and not simply refreshing the integrated data repository 1104 when new data is available, the integrated data repository 1104 enhances privacy protection with respect to data stored by the integrated data repository 1104. That is, in situations where data repositories are refreshed simply with new data, it may be possible to more easily track individuals associated with data that has been newly added to a data repository because the number of new individuals added at a given time is typically smaller than an existing number of individuals that already have data stored by the data repository.

In various examples, data stored by the integrated data repository 1104 may be accessed via a database management system. In addition, the integrated data repository 1104 may store data according to one or more database models. In one or more examples, the integrated data repository 1104 may store data according to one or more relational database technologies. For example, the integrated data repository 1104 may store data according to a relational database model. In one or more additional examples, the integrated data repository 1104 may store data according to an object-oriented database model. In one or more further examples, the integrated data repository 1104 may store data according to an extensible markup language (XML) database model. In still additional examples, the integrated data repository 1104 may store data according to a structured query language (SQL) database model. In still further examples, the integrated data repository may store data according to an image database model.

The data integration system 1114 may generate the integrated data repository 1104 by generating a number of data tables and creating links between the data tables. The links may indicate logical couplings between the data tables. The data integration system 1114 may generate the data tables by extracting specified sets of data from the information obtained from the data repositories 1106, 1108, 1110, 1112 and storing the data in rows and columns of respective data tables. In various examples, the logical couplings between data tables may include at least one of a one-to-one link where a row of information in one data table corresponds to a row of information in another data table, a one-to-many link where a row of information in one data table corresponds to multiple rows of information in another data table, or a many-to-many link where multiple rows of information of one data table correspond to multiple rows of information in another data table.

The number of data tables may be arranged according to a data repository schema 1116. In the illustrative example of FIG. 1, the data repository schema 1114 includes a first data table 1118, a second data table 1120, a third data table 1122, a fourth data table 1124, and a fifth data table 1124. Although the illustrative example of FIG. 1 includes five data tables, in additional implementations, the data repository schema 1116 may include more data tables or fewer data tables. The data repository schema 1116 may also include links between the data tables 1118, 1120, 1122, 1124, 1128. The links between the data tables 1118, 1120, 1122, 1124, 1126 may indicate that information retrieved from one of the data tables 1118, 1120, 1122, 1124, 1126 results in additional information stored by one or more additional data tables 1118, 1120, 1122, 1124, 1126 to be retrieved. Additionally, not all the data tables 1118, 1120, 1122, 1124, 1126 may be linked to each of the other data tables 1118, 1120, 1120, 1122, 1124, 1126. In the illustrative example of FIG. 1, the first data table 1118 is logically coupled to the second data table 1118 by a first link 1128 and the first data table 1118 is logically coupled to the fourth data table 1124 by a second link 1130. In addition, the second data table 1120 is logically coupled to the third data table 1122 via a third link 1132 and the fourth data table 1124 is logically coupled to the fifth data table 1126 via a fourth link 1134. Further, the third data table 1122 is logically coupled to the fifth data table 1126 via a fifth link 1136.

In various examples, as data tables are added to and/or removed from the data repository schema 1116, additional links between data tables may be added to or removed from the data repository schema 1116. In one or more illustrative examples, the integrated data repository 1104 may store data tables according to the data repository schema 1116 for at least a portion of the individuals for which the data integration system 1114 obtained information from a combination of at least two of the health insurance claims data repository 1106, the molecular data repository 1108, the one or more additional data repositories 1110, and the one or more reference information data repositories 1112. As a result, the integrated data repository 1104 may store respective instances of the data tables 1118, 1120, 1122, 1124, 1126 according to the data repository schema 1116 for thousands, tens of thousands, up to hundreds of thousands or more individuals.

The data integration and analysis system 1102 may also include a data pipeline system 1138. The data pipeline system 1138 may include a number of algorithms, software code, scripts, macros, or other bundles of computer-executable instructions that process information stored by the integrated data repository 1104 to generate additional datasets. The additional datasets may include information obtained from one or more of the data tables 1118, 1120, 1122, 1124, 1126. The additional datasets may also include information that is derived from data obtained from one or more of the data tables 1118, 1120, 1122, 1124, 1126. The components of the data pipeline system 1138 implemented to generate a first additional dataset may be different from the components of the data pipeline system 1138 used to generate a second additional dataset.

In one or more examples, the data pipeline system 1138 may generate a dataset that indicates pharmacy treatments received by a number of individuals. In one or more illustrative examples, the data pipeline system 1138 may analyze information stored in at least one of the data tables 1118, 1120, 1122, 1124, 1126 to determine health insurance codes corresponding to pharmaceutical treatments received by a number of individuals. The data pipeline system 1138 may analyze the health insurance codes corresponding to pharmaceutical treatments with respect to a library of data that indicates specified pharmaceutical treatments that correspond to one or more health insurance codes to determine names of pharmaceutical treatments that have been received by the individuals. In one or more additional examples, the data pipeline system 1138 may analyze information stored by the integrated data repository 1104 to determine medical procedures received by a number of individuals. To illustrate, the data pipeline system 1138 may analyze information stored by one of the data tables 1118, 1120, 1122, 1124, 1126 to determine treatments received by individuals via at least one of injection or intravenously. In one or more further examples, the data pipeline system 1138 may analyze information stored by the integrated data repository 1104 to determine episodes of care for individuals, lines of therapy received by individuals, progression of a biological condition, or time to next treatment. In various examples, the datasets generated by the data pipeline system 1138 may be different for different biological conditions. For example, the data pipeline system 1138 may generate a first number of datasets with respect to a first type of cancer, such as lung cancer, and a second number of datasets with respect to a second type of cancer, such as colorectal cancer.

The data pipeline system 1138 may also determine one or more confidence levels to assign to information associated with individuals having data stored by the integrated data repository 1104. The respective confidence levels may correspond to different measures of accuracy for information associated with individuals having data stored by the integrated data repository 1104. The information associated with the respective confidence levels may correspond to one or more characteristics of individuals derived from data stored by the integrated data repository 1104. Values of confidence levels for the one or more characteristics may be generated by the data pipeline system 1138 in conjunction with generating one or more datasets from the integrated data repository 1104. In one or more examples, a first confidence level may correspond to a first range of measures of accuracy, a second confidence level may correspond to a second range of measures of accuracy, and a third confidence level may correspond to a third range of measures of accuracy. In one or more additional examples, the second range of measures of accuracy may include values that are less values of the first range of measures of accuracy and the third range of measures of accuracy may include values that are less than values of the second range of measures of accuracy. In one or more illustrative examples, information corresponding to the first confidence level may be referred to as Gold standard information, information corresponding to the second confidence level may be referred to as Silver standard information, and information corresponding to the third confidence level may be referred to as Bronze standard information.

The data pipeline system 1138 may determine values for the confidence levels of characteristics of individuals based on a number of factors. For example, a respective set of information may be used to determine characteristics of individuals. The data pipeline system 1138 may determine the confidence levels of characteristics of individuals based on an amount of completeness of the respective set of information used to determine a characteristic for an individual. In situations where one or more pieces of information are missing from the set of information associated with a first number of individuals, the confidence levels for a characteristic may be lower than for a second number of individuals where information is not missing from the set of information. In one or more examples, an amount of missing information may be used by the data pipeline system 1138 to determine confidence levels of characteristics of individuals. To illustrate, a greater amount of missing information used to determine a characteristic of an individual may cause confidence levels for the characteristic to be lower than in situations where the amount of missing information used to determine the characteristic is lower. Further, different types of information may correspond to various confidence levels for a characteristic. In one or more examples, the presence of a first piece of information used to determine a characteristic of an individual may result in confidence levels for the characteristic being higher than the presence of a second piece of information used to determine the characteristic.

In one or more illustrative examples, the data pipeline system 1138 may determine a number of individuals included in a cohort with a primary diagnosis of lung cancer (or other biological condition). The data pipeline system 1138 may determine confidence levels for respective individuals with respect to being classified as having a primary diagnosis of lung cancer. The data pipeline system 1138 may use information from a number of columns included in the data tables 1118, 1120, 1122 1124, 1126 to determine a confidence level for the inclusion of individuals within a lung cancer cohort. The number of columns may include health insurance codes related to diagnosis of biological conditions and/or treatments of biological conditions. Additionally, the number of columns may correspond to dates of diagnosis and/or treatment for biological conditions. The data pipeline system 1138 may determine that a confidence level of an individual being characterized as being part of the lung cancer cohort is higher in scenarios where information is available for each of the number of columns or at least a threshold number of columns than in instances where information is available for less than a threshold number of columns. Further, the data pipeline system 1138 may determine confidence levels for individuals included in a lung cancer cohort based on the type of information and availability of information associated with one or more columns. To illustrate, in situations where one or more diagnosis codes are present in relation to one or more periods of time for a group of individuals and one or more treatment codes are absent, the data pipeline system 1138 may determine that the confidence level of including the group of individuals in the lung cancer cohort is greater than in situations where at least one of the diagnosis codes is absent and the treatment codes used to determine whether individuals are included in the lung cancer cohort are present.

The data integration and analysis system 1102 may include a data analysis system 1140. The data analysis system 1140 may receive integrated data repository requests 1142 from one or more computing devices, such as an example computing device 1144. The one or more integrated data repository requests 1142 may cause data to be retrieved from the integrated data repository 1104. In various examples, the one or more integrated data repository requests 1142 may cause data to be retrieved from one or more datasets generated by the data pipeline system 1138. The integrated data repository requests 1142 may specify the data to be retrieved from the integrated data repository 1104 and/or the one or more datasets generated by the data pipeline system 1138. In one or more additional examples, the integrated data repository requests 1142 may include one or more prebuilt queries that correspond to computer-executable instructions that retrieve a specified set of data from the integrated data repository 1104 and/or one or more datasets generated by the data pipeline system 1138.

In response to one or more integrated data repository requests 1142, the data analysis system 1140 may analyze data retrieved from at least one of the integrated data repository 1104 or one or more datasets generated by the data pipeline system 1138 to generate data analysis results 1146. The data analysis results 1146 may be sent to one or more computing devices, such as example computing device 1148. Although the illustrative example of FIG. 1 shows that the one or more integrated data repository requests 1142 from one computing device 1144 and the data analysis results 1146 being sent to another computing device 1148, in one or more additional implementations, the data analysis results 1146 may be received by a same computing device that sent the one or more integrated data repository requests 1142. The data analysis results 1146 may be displayed by one or more user interfaces rendered by the computing device 1144 or the computing device 1148.

In one or more examples, the data analysis system 1140 may implement at least one of one or more machine learning techniques or one or more statistical techniques to analyze data retrieved in response to one or more integrated data repository requests 1142. In one or more examples, the data analysis system 1140 may implement one or more artificial neural networks to analyze data retrieved in response to one or more integrated data repository requests 1142. To illustrate, the data analysis system 1140 may implement at least one of one or more convolutional neural networks or one or more residual neural networks to analyze data retrieved from the integrated data repository 1104 in response to one or more integrated data repository requests 1142. In at least some examples, the data analysis system 1140 may implement one or more random forests techniques, one or more support vector machines, or one or more Hidden Markov models to analyze data retrieved in response to one or more integrated data repository requests 1142. One or more statistical models may also be implemented to analyzed data retrieved in response to one or more integrated data repository requests 1142 to identify at least one of correlations or measures of significance between characteristics of individuals. For example, log rank tests may be applied to data retrieved in response to one or more integrated data repository requests 1142. In addition, Cox proportional hazards models may be implemented with respect to date retrieved in response to one or more integrated data repository requests 1142. Further, Wilcoxon singed rank tests may be applied to data retrieved in response to one or more integrated data repository requests 1142. In still other examples, a z-score analysis may be performed with respect to data retrieved in response to one or more integrated data repository requests 1142. In still additional examples, a Kaplan Meier analysis may be performed with respect to data retrieved in response to one or more integrated data repository requests 1142. In at least some examples, one or more machine learning techniques may be implemented in combination with one or more statistical techniques to analyze data retrieved in response to one or more integrated data repository requests 1142.

In one or more illustrative examples, the data analysis system 1140 may determine a rate of survival of individuals in which lung cancer is present in response to one or more treatments. In one or more additional illustrative examples, the data analysis system 1140 may determine a rate of survival of individuals having one or more genomic region mutations in which lung cancer is present in response to one or more treatments. In various examples, the data analysis system 1140 may generate the data analysis results 1146 in situations where the data retrieved from at least one of the integrated data repository 1104 or the one or more datasets generated by the data pipeline system 1138 satisfies one or more criteria. For example, the data analysis system 1140 may determine whether at least a portion of the data retrieved in response to one or more integrated data repository requests 1142 satisfies a threshold confidence level. In situations where the confidence level for at least a portion of the date retrieved in response to one or more integrated data repository requests 1142 is less than a threshold confidence level, the data analysis system 1140 may refrain from generating at least a portion of data analysis results 1146. In scenarios where the confidence level for at least a portion of the data retrieved in response to one or more integrated data repository requests 1142 is at least a threshold confidence level, the data analysis system 1140 may generate at least a portion of the data analysis results 1146. In various examples, the threshold confidence level may be related to the type of data analysis results 1146 being generated by the data analysis system 1140.

In one or more illustrative examples, the data analysis system 1140 may receive an integrated data repository request 1142 to generate data analysis results 1146 that indicate a rate of survival of one or more individuals. In these instances, the data analysis system 1140 may determine whether the data stored by the integrated data repository 1104 and/or by one or more datasets generated by the data pipeline system 1138 satisfies a threshold confidence level, such as a Gold standard confidence level. In one or more additional examples, the data analysis system 1140 may receive an integrated data repository request 1142 to generate data analysis results 1146 that indicate a treatment received by one or more individuals. In these implementations, the data analysis system 1140 may determine whether the data stored by the integrated data repository 1104 and/or by one or more datasets generated by the data pipeline system 1138 satisfies a lower threshold confidence level, such as a Bronze standard confidence level.

In one or more additional illustrative examples, the data analysis system 1140 may receive an integrated data repository request 1142 to determine individuals having one or more genomic mutations and that have received one or more treatments for a biological condition. Continuing with this example, the data analysis system 1140 can determine a survival rate of individuals with the one or more genomic mutations in relation to the one or more treatments received by the individuals. The data analysis system 1140 can then identify based on the survival rate of individuals an effectiveness of treatments for the individuals in relation to genomic mutations that may be present in the individuals. In this way, health outcomes of individuals may be improved by identifying prospective treatments that may be more effective for populations of individuals having one or more genomic mutations than current treatments being provided to the individuals.

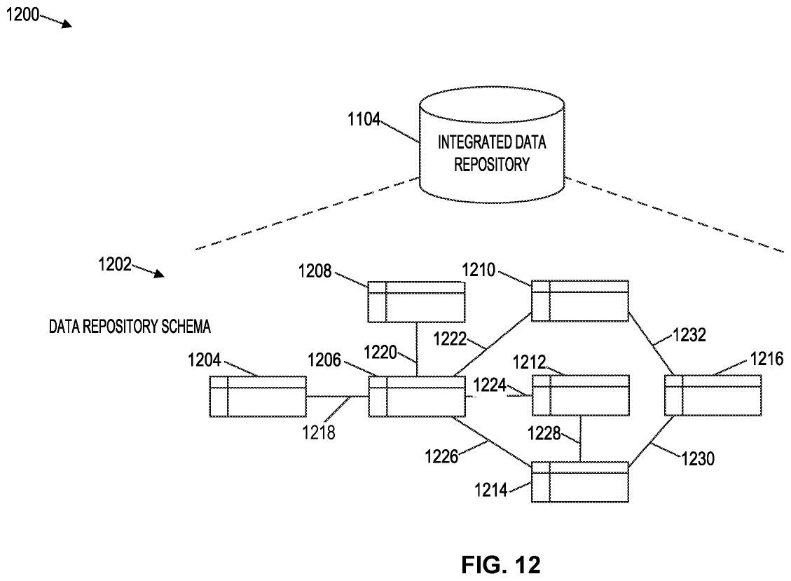

FIG. 12 illustrates an example framework 1200 corresponding to an arrangement of data tables in an integrated data repository, according to one or more implementations. In the illustrative example of FIG. 12, the framework 1200 includes a data repository schema 1202 that includes a first data table 204, a second data table 1206, a third data table 1208, a fourth data table 1210, a fifth data table 1212, a sixth data table 1214, and a seventh data table 1216. Although the illustrative example of FIG. 2 includes seven data tables, in additional implementations, the data repository schema 1202 may include more data tables or fewer data tables. The data repository schema 1202 may also include links between the data tables 204, 1206, 1208, 1210, 1212, 1214, 1216. The links between the data tables 204, 1206, 1208, 1210, 1212, 1214, 1216 may indicate that information retrieved from one of the data tables 204, 1206, 1208, 1210, 1212, 1214, 1216 results in additional information stored by one or more additional data tables 204, 1206, 1208, 1210, 1212, 1214, 1216 to be retrieved. Additionally, not all the data tables 204, 1206, 1208, 1210, 1212, 1214, 1216 may be linked to each of the other data tables 204, 1206, 1208, 1210, 1212, 1214, 1216. In the illustrative example of FIG. 2, the first data table 204 is logically coupled to the second data table 1206 by a first link 1218 and the third data table 1208 is logically coupled to the second data table 1206 by a second link 1220. The second data table 1206 is also logically coupled to the fourth data table 1210 by a third link 1222, the second data table 1206 is logically coupled to the fifth data table 1212 by a fourth link 1224, and the second data table 1206 is logically coupled to the sixth data table 1214 by a fifth link 1226. In addition, fifth data table 1212 is logically coupled to the sixth data table 1214 by a sixth link 1228 and the sixth data table 1214 is logically coupled to the seventh data table 1216 by a seventh link 1230. Further, the seventh data table 1216 is logically coupled to the fourth data table 1210 by an eighth link 1232. In various examples, as data tables are added to and/or removed from the data repository schema 1202, additional links between data tables may be added to or removed from the data repository schema 1202. In one or more illustrative examples, the integrated data repository 1104 may store data tables according to the data repository schema 1202 for at least a portion of the individuals for which the data integration system 1114 obtained information from a combination of at least two of the health insurance claims data repository 1106, the molecular data repository 1108, and the one or more additional data repositories 1110. As a result, the integrated data repository 1104 may store respective instances of the data tables 204, 1206, 1208, 1210, 1212, 1214, 1216 according to the data repository schema 204 for thousands, tens of thousands, up to hundreds of thousands or more individuals.

In one or more examples, the first data table 204 may store data corresponding to genomics and genomics testing for individuals. For example, the first data table 204 may include columns that include information corresponding to a panel used to generate genomics data, mutations of genomic regions, types of mutations, copy numbers of genomic regions, coverage data indicating numbers of nucleic acid molecules identified in a sample having one or more mutations, testing dates, and patient information. The first data table 204 may also include one or more columns that include health insurance data codes that may correspond to one or more diagnosis codes. Additionally, the information in first data table 204 may include at least one identifier for an individual that is associated with an instance of the first data table 204.

The second data table 1206 may store data related to one or more patient visits by individuals to one or more healthcare providers. The third data table 1208 may store information corresponding to respective services provided to individuals with respect to one or more patient visits to one or more healthcare providers indicated by the second data table 1206. To illustrate, an individual may visit a healthcare provider and multiple services may be performed with respect to the individual at the visit. A second data table 1206 may include columns indicating information for each of the multiple services performed during the patient visit. Multiple third data tables 1208 may be generated with respect to the patient visit that include columns indicating information on a more granular level for a respective service provided during the patient visit than the information stored by the second data table 1206 related to the patient visit. For example, the second data table 1206 may include multiple columns indicating a health insurance code for different services provided to an individual during a patient visit and a third data table 1208 related to one of the services may include multiple columns for additional health insurance codes that correspond to additional information related to the respective services. The second data table 1206 and the third data table(s) 1208 for a patient visit may indicate one or more dates of service corresponding to the patient visit.

The fourth data table 1210 may include columns that indicate information about individuals for which information is stored by the integrated data repository 1104. For example, the fourth data table 1210 may include columns that indicate information related to at least one of a location of an individual, a gender of an individual, a date of birth of an individual, a date of death of an individual (if applicable), or one or more keys associated with the individual. In one or more examples, the fourth data table 1210 may include one or more columns related to whether erroneous data has been identified for an individual. In various examples, a single fourth data table 1210 may be generated for respective individuals. Thus, the data repository schema 1202 may include multiple instances of the fourth data table 1210, such as thousands, tens of thousands, up to hundreds of thousands or more.

The fifth data table 1212 may include columns that indicate information related to a health insurance company or governmental entity that made payment for one or more services provided to respective individuals. For example, the fifth data table 1212 may include one or more payer identifiers. The sixth data table 1214 may include columns that include information corresponding to health insurance coverage information for respective individuals. In one or more examples, the sixth data table 1214 may include columns indicating the presence of medical coverage for an individual, the presence of pharmacy coverage for an individual, and a type of health insurance plan related to the individual, such as health maintenance organization (HMO), preferred provider organization (PPO), and the like.

The seventh data table 1216 may include columns that indicate information related to pharmaceutical treatments obtained by a respective individual. In one or more examples, the seventh data table 1216 may include one or more columns indicating health insurance codes corresponding to pharmaceutical treatments that are available via a pharmacy. The health insurance codes may correspond to individual pharmaceutical treatments. Additionally, the health insurance codes may indicate a diagnosis of a biological condition with respect to an individual. The seventh data table 1216 may also include additional information, such as at least one of dosage amounts, number of days' supply, quantity dispensed, number of refills authorized, dates of service, or information related to the individual receiving the pharmaceutical treatment.

In various examples, the data repository schema 1202 may provide results of analysis of the information stored by the data tables 204, 1206, 1208, 1210, 1212, 1214, 1216 in a more efficient manner than typical data repository schemas. For example, the logical connections between the data tables 204, 1206, 1208, 1210, 1212, 1214, 1216 are arranged to efficiently retrieve data that is related across the different data tables 204, 1206, 1208, 1210, 1212, 1214, 1216. In situations where the data tables 204, 1206, 1208, 1210, 1212, 1214, 1216 are arranged in a serial manner and/or in situations where a greater number of the data tables 204, 1206, 1208, 1210, 1212, 1214, 1216 are logically connected, retrieving data from the integrated data repository 1104 from one or more of the data tables 204, 1206, 1208, 1210, 1212, 1214, 1216 to responds to a request for information from the integrated data repository 1104 will be less efficient than in situations where the data repository schema 1202 is implemented.

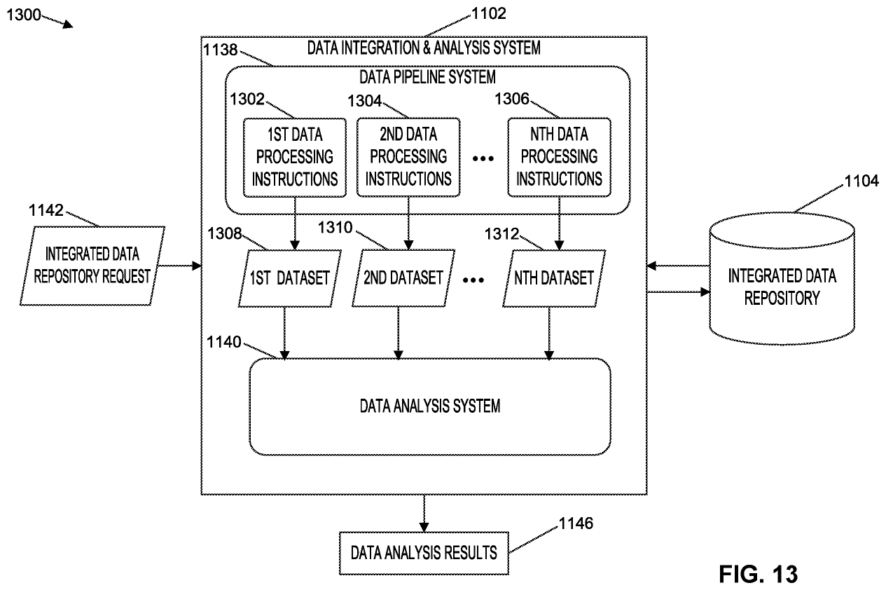

FIG. 13 illustrates an architecture 1300 to generate one or more datasets from information retrieved from a data repository that integrates health related data from a number of sources, according to one or more implementations. The architecture 1300 may include the data integration and analysis system 1102 and the integrated data repository 1104. Additionally, the data integration and analysis system 1102 may include at least the data pipeline system 1138 and the data analysis system 1140. The data pipeline system 1138 may include a number of sets of data processing instructions that are executable to generate respective datasets that may be analyzed by the data analysis system 1140 in response to an integrated data repository request 1142 to generate data analysis results 1146.

The data pipeline system 1138 may include first data processing instructions 1302, second data processing instructions 1304, up to Nth data processing instructions 1306. The data processing instructions 1302, 1304, 1306 may be executable by one or more processing units to perform a number of operations to generate respective datasets using information obtained from the integrated data repository 1104. In one or more illustrative examples, the data processing instructions 1302, 1304, 1306 may include at least one of software code, scripts, API calls, macros, and so forth. The first data processing instructions 1302 may be executable to generate a first dataset 1308. In addition, the second data processing instructions 1304 may be executable to generate a second dataset 1310. Further, the Nth data processing instructions 1306 may be executable to generate an Nth dataset 1312. In various examples, after the data integration and analysis system 1102 generates the integrated data repository 1104, the data pipeline system 1138 may cause the data processing instructions 1302, 1304, 1306 to be executed to generate the datasets 1308, 1310, 1312. In one or more examples, the datasets 1308, 1310, 1312 may be stored by the integrated data repository 1104 or by an additional data repository that is accessible to the data integration and analysis system 1102. At least a portion of the data processing instructions 1302, 1304, 1306 may analyze health insurance codes to generate at least a portion of the datasets 1308, 1310, 1312. Additionally, at least a portion of the data processing instructions 1302, 1304, 1306 may analyze genomics data to generate at least a portion of the datasets 1308, 1310, 1312.

In one or more examples, the first data processing instructions 1302 may be executable to retrieve data from one or more first data tables stored by the integrated data repository 1104. The first data processing instructions 1302 may also be executable to retrieve data from one or more specified columns of the one or more first data tables. In various examples, the first data processing instructions 1302 may be executable to identify individuals that have a health insurance code stored in one or more column and row combinations that correspond to one or more diagnosis codes. The first data processing instructions 1302 may then be executable to analyze the one or more diagnosis codes to determine a biological condition for which the individuals have been diagnosed. In one or more illustrative examples, the first data processing instructions 1302 may be executable to analyze the one or more diagnosis codes with respect to a library of diagnosis codes that indicates one or more biological conditions that correspond to respective diagnosis codes. The library of diagnosis codes may include hundreds up to thousands of diagnosis codes. The first data processing instructions 1302 may also be executable to determine individuals diagnosed with a biological condition by analyzing timing information of the individuals, such as dates of treatment, dates of diagnosis, dates of death, one or more combinations thereof, and the like.

The second data processing instructions 1304 may be executable to retrieve data from one or more second data tables stored by the integrated data repository 1104. The second data processing instructions 1304 may also be executable to retrieve data from one or more specified columns of the one or more second data tables. In various examples, the second data processing instructions 1304 may be executable to identify individuals that have a health insurance code stored in one or more column and row combinations that correspond to one or more treatment codes. The one or more treatment codes may correspond to treatments obtained from a pharmacy. In one or more additional examples, the one or more treatment codes may correspond to treatments received by a medical procedure, such as an injection or intravenously. The second data processing instructions 1304 may be executable to determine one or more treatments that correspond to the respective health insurance codes included in the one or more second data tables by analyzing the health insurance code in relation to a predetermined set of information. The predetermined set of information may include a data library that indicates one or more treatments that correspond to one out of hundreds up to thousands of health insurance codes. The second data processing instructions 1304 may generate the second dataset 1310 to indicate respective treatments received by a group of individuals. In one or more illustrative examples, the group of individuals may correspond to the individuals included in the first dataset 1308. The second dataset 1310 may be arranged in rows and columns with one or more rows corresponding to a single individual and one or more columns indicating the treatments received by the respective individual.

The Nth processing instructions 1306 (where N may be any positive integer) may be executable to generate the Nth dataset 1312 by combining information from a number of previously generated datasets, such as the first dataset 1308 and the second dataset 1310. In addition, the Nth processing instructions 1306 may be executable to generate the Nth dataset 1312 to retrieve additional information from one or more additional columns of the integrated data repository 1104 and incorporate the additional information from the integrated data repository 1104 with information obtained from the first dataset 1308 and the second dataset 1310. For example, the Nth processing instructions 1306 may be executable to identify individuals included in the first dataset 1308 that are diagnosed with a biological condition and analyze specified columns of one or more additional data tables of the integrated data repository 1104 to determine dates of the treatments indicated in the second dataset 1210 that correspond to the individuals included in the first dataset 1308. In one or more further examples, the Nth processing instructions 1306 may be executable to analyze columns of one or more additional data tables of the integrated data repository 1104 to determine dosages of treatments indicated in the second dataset 1310 received by the individuals included in the first dataset 1308. In this way, the Nth processing instructions 1306 may be executable to generate an episodes of care dataset based on information included in a cohort dataset and a treatments dataset.

In one or more illustrative examples, in response to receiving an integrated data repository request 1142, the data analysis system 1140 may determine one or more datasets that correspond to the features of the query related to the integrated data repository request 1142. For example, the data analysis system 1140 may determine that information included in the first dataset 1308 and the second dataset 1310 is applicable to responding to the integrated data repository request 1142. In these scenarios, the data analysis system 1140 may analyze at least a portion of the data included in the first dataset 1308 and the second dataset 1310 to generate the data analysis results 1146. In one or more additional examples, the data analysis system 1140 may determine different datasets to respond to different queries included in the integrated data repository request 1142 in order to generate the data analysis results 1146.

The use of specific sets of data processing instructions to generate respective data sets may reduce the number of inputs from users of the data integration and analysis system 1102 as well as reduce the computational load, such as the amount of processing resources and memory, utilized to process integrated data repository requests 1142. For example, without the specific architecture of the data pipeline system 1138, each time an integrated data repository request 1142 is received, the data utilized to respond to the integrated data repository request 1142 is assembled from the data repository 1104. In contrast, by implementing the data pipeline system 1138 to execute the data processing instruction 1302, 1304, 1306 to generate the datasets 1308, 1310, 1312, the data needed to respond to various integrated data repository requests 1142 has already been assembled and may be accessed by the data analysis system 1140 to respond to the integrated data repository request 1142. Thus, the computing resources used to respond to the integrated data repository request 1142 by implementing the data pipeline system 1138 to generate the datasets 1308, 1310, 1312 are less than typical systems that perform an information parsing and collecting process for each integrated data repository request 1142. Further, in situations where the data pipeline system 1138 has not been implemented, users of the data integration and analysis system 1102 may need to submit multiple integrated data repository request 1142 in order to analyze the information that the users are intending to have analyzed either because the ad hoc collection of data to respond to an integrated data repository request 1142 in typical systems is inaccurate or because the data analysis system 1140 is called upon multiple times to perform an analysis of information in typical systems that may be performed using a single integrated data repository request 1142 when the data pipeline system 1138 is implemented.

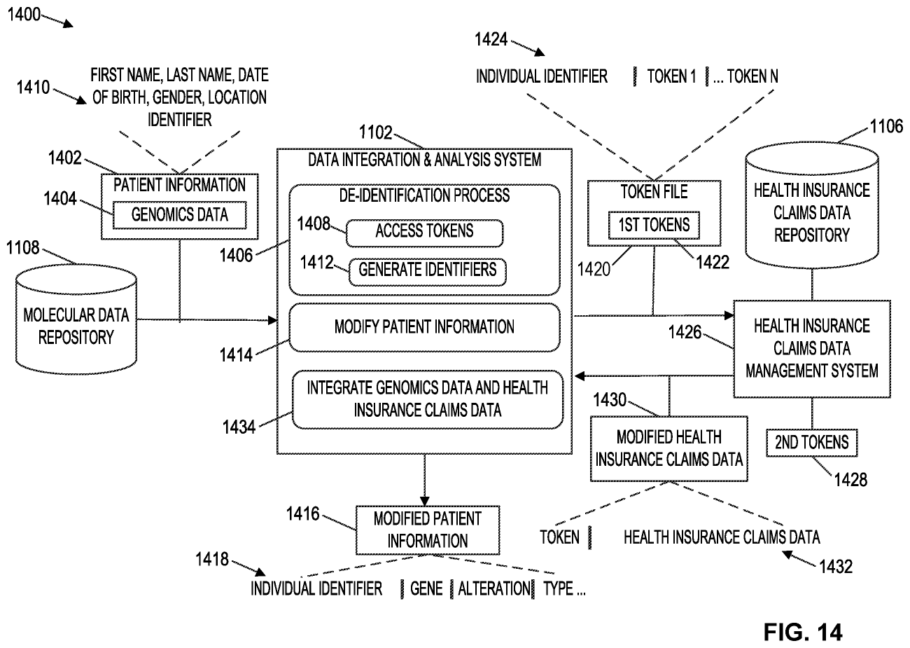

FIG. 14 illustrates an architecture 1400 to generate an integrated data repository that includes de-identified health insurance claims data and de-identified genomics data it, according to one or more implementations. The architecture 1400 may include the data integration and analysis system 1102, the health insurance claims data repository 1106, and the molecular data repository 1108. The data integration and analysis system 1102 may obtain patient information 1402 from the molecular data repository 1108. The patient information 1402 may include genomics data 1404 for individuals having data stored by the molecular data repository 1108. The genomics data 1404 may indicate results of one or more nucleic acid sequencing operations that analyze sequences of nucleic acid molecules included in a sample obtained from the individuals with respect to one or more target genomic regions. In one or more examples, the sample may be obtained from tissue of one or more individuals. In one or more additional examples, the sample may be obtained from fluid of one or more individuals, such as blood or plasma. The one or more target genomic regions may correspond to genomic regions that correspond to the presence of one or more biological conditions. For example, the target regions may correspond to genomic regions of a reference genome having mutations that are present in individuals in which a biological condition is present. In one or more illustrative examples, the target regions may correspond to genomic regions of a reference human genome in which one or more mutations are present in individuals in which one or more forms of cancer are present. The patient information 1402 may also include information indicating personal information about individuals with data stored by the molecular data repository 1108 and information corresponding to the testing and analysis performed on samples provided by individuals.

The data integration and analysis system 1102 may perform a de-identification process 1406 that anonymizes personal information obtained from the molecular data repository 1108. The data integration and analysis system 1102 may implement one or more computational techniques as part of the de-identification process to anonymize data related to individuals stored by the molecular data repository 1108 such that the de-identified data protects the privacy of the individuals and is in compliance with one or more privacy regulation frameworks. The de-identification process 1406 may include, at 1408, accessing tokens. In various examples, the tokens may comprise an alphanumeric string of characters. In one or more examples, the tokens may be generated by the data integration and analysis system 1102. In one or more additional examples the tokens may be generated by a third-party and obtained by the data integration and analysis system 1102.

The tokens may be generated using one or more hash functions in relation to a subset 1410 of the patient information 1402. To illustrate, for individuals that have information stored by the molecular data repository 1108, the tokens may be generated using a combination of at least a portion of a first name of the respective individuals, at least a portion of the last name of the respective individuals, at least a portion of a date of birth of the respective individuals, a gender of the individuals, and at least a portion of a location identifier of the respective individuals. The de-identification process 1406 may also include, at 1412, generating identifiers for individuals that have data stored by the molecular data repository 1108. The identifiers may be generated by the data integration and analysis system 1102 using one or more hash functions that are different from the one or more hash functions used to generate the tokens. In one or more illustrative examples, the data integration and analysis system 1102 may generate an intermediate version of respective identifiers using one or more hash function and then apply one or more salting techniques to the intermediate versions of the identifiers to generate final versions of the identifiers. A salt function comprises a function configured to add at least one random bit to each intermediate identifier to generate a respective final identifier. In various examples, the data integration and analysis system 1102 may generate the identifiers at 1412 using at least a portion of the information for respective individuals stored by the molecular data repository 1108. In one or more illustrative examples, the identifiers may be generated based on a patient identifier included in the patient information 1402. The identifiers generated by the data integration and analysis system 1102 may be unique for respective individuals having data stored by the molecular data repository 1108.

At operation 1414, the data integration and analysis system 1102 may generate modified patient information 1416 based on the identifiers. The modified patient information 1416 may include genomics data 1404 related to individuals associated with the molecular data repository 1108 and the identifiers of the respective individuals. The modified patient information 1416 may have a data structure 1418. The data structure 1418 may include a column that includes respective identifiers of individuals associated with the molecular data repository 1108 and a number of columns that include genomics data 1404 related to the individuals, such as identifiers of one or more genes, alterations to the one or more genes, type of alteration to the genes, and so forth.

The data integration and analysis system 1102 may generate a token file 1420. The token file 1420 may include first tokens 1422 accessed at operation 1408 for respective individuals having data stored by the molecular data repository 1108. The token file 1420 may have a data structure 11424 that includes a number of columns that include information for respective individuals. The data structure 11424 may include a column indicating respective identifiers generated by the data integration and analysis system 1102 and columns indicating one or more first tokens 1422 associated with the respective identifiers. The data integration and analysis system 1102 may send the token file 1420 to a health insurance claims data management system 1426 that is coupled to the health insurance claims data repository 1106. The health insurance claims data management system 1426 may analyze the first tokens 1422 with respect to corresponding second tokens 1428. The second tokens 1428 may be accessed by or generated by the health insurance claims data management system 1426. The second tokens 1428 may be generated using a same or similar subset of information for individuals having data stored in the health insurance claims data repository 1106 as the subset 1410 of the patient information 1402. For example, the second tokens 1428 may be generated using a combination of at least a portion of a first name of the respective individuals, at least a portion of the last name of the respective individuals, at least a portion of a date of birth of the respective individuals, a gender of the individuals, and at least a portion of a location identifier of the respective individuals.

In various examples, the health insurance claims data management system 1426 may retrieve health insurance claims data from the health insurance claims data repository 1106 for individuals associated with respective second tokens 1428 that match corresponding first tokens 1422. A first token 1422 may match a second token 1428 when the data of the first token 1422 has at least a threshold amount of similarity with respect to the data of the second token 1428. In one or more examples, a first token 1422 may match a second token 1428 when the data of the first token 1422 is the same as the data of the second token 1428.

In response to identifying health insurance claims data for individuals having respective second tokens 1428 that correspond to a respective first token 1422, the health insurance claims data management system 1426 may generate modified health insurance claims data 1430. The health insurance claims data management system 1426 may send the modified health insurance claims data 1430 to the data integration and analysis system 1102. In one or more examples, the modified health insurance claims data 1430 may be formatted according to a data structure 1432. The data structure 1432 may include a column that includes a subset of the second tokens 1428 that correspond to the first tokens 1422 and a number of columns that include the health insurance claims data.

At operation 1434, the data integration and analysis system 1102 may integrate genomics data and health insurance claims data of individuals that are common to both the molecular data repository 1108 and the health insurance claims data repository 1106. The data integration and analysis system 1102 may determine individuals that are common to both the molecular data repository 1108 and the health insurance claims data repository 1106 by determining genomics data and health insurance claims data corresponding to common tokens. The data integration and analysis system 1102 may determine that a first token 1422 related to a portion of the genomics data 1404 corresponds to a second token 1428 related to a portion of the health insurance claims data by determining a measure of similarity between the first token 1422 and the second token 1428. In scenarios where the first token 1422 has at least a threshold amount of similarity with respect to the second token 1428, the data integration and analysis system 1102 may store the corresponding portion of the genomics data 1404 and the corresponding portion of the health insurance claims data in relation to the identifier of the individual in an integrated data repository, such as the integrated data repository 1104 of FIG. 1, FIG. 2, and FIG. 3.

The implementation of the architecture 1400 may implement a cryptographic protocol that enables de-identified information from disparate data repositories to be integrated into a single data repository. In this way, the security of the data stored by the integrated data repository 1104 is increased. Additionally, the cryptographic protocol implemented by the architecture 1400 may enable more efficient retrieval and accurate analysis of information stored by the integrated data repository 1104 than in situations where the cryptographic protocol of the architecture 1400 is not utilized. For example, by generating a token file 1420 that includes first tokens 1422 using a cryptographic technique based on a specified set of information stored by the molecular data repository 1104 and utilizing second tokens 1428 generated using a same or similar cryptographic technique with respect to the similar or same set of information stored by the health insurance claims data repository 1106, the data integration and analysis system 1102 may match information stored by disparate data repositories that correspond to a same individual. Without implementing the cryptographic protocol of the architecture 1400, the probability of incorrectly attributing information from one data repository to one or more individuals increases, which decreases the accuracy of results provided by the data integration and analysis system 1102 in response to integrated data repository requests 1142 sent to the data integration and analysis system 1102.

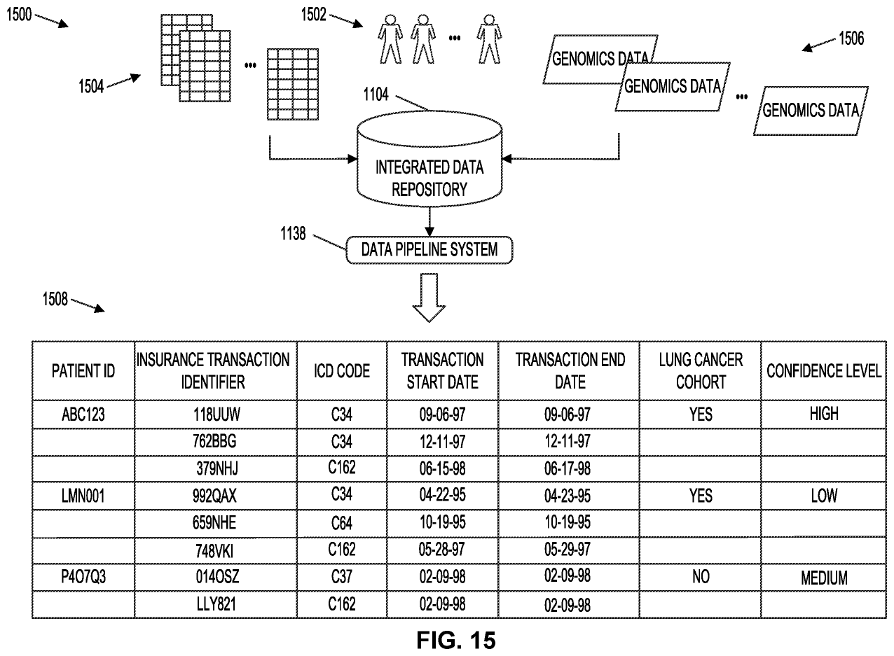

FIG. 15 illustrates a framework 1500 to generate a dataset, by a data pipeline system 1138, based on data stored by an integrated data repository 1104, according to one or more implementations. The integrated data repository 1104 may store health insurance claims data and genomics data for a group of individuals 1502. For example, the integrated data repository 1104 may store information obtained from health insurance claims records 1504 of the group of individuals 1502. For each individual included in the group of individuals 1502, the integrated data repository 1104 may store information obtained from multiple health insurance claim records 1504. In various examples, the information stored by the integrated data repository 1104 may include and/or be derived from thousands, tens of thousands, hundreds of thousands, up to millions of health insurance claims records 1504 for a number of individuals. Additionally, each health insurance claim record may include multiple columns. As a result, the integrated data repository 1104 may be generated through the analysis of millions of columns of health insurance claims data.

Further, although the health insurance claims data may be organized according to a structured data format, health insurance claims data is typically arranged to be viewed by health insurance providers, patients, and healthcare providers in order to show financial information and insurance code information related to services provided to individuals by healthcare providers. Thus, health insurance claims data is not easily analyzed to gain insights that may be available in relation to characteristics of individuals in which a biological condition is present and that may aid in the treatment of the individuals with respect to the biological condition. The integrated data repository 1104 may be generated and organized by analyzing and modifying raw health insurance claims data in a manner that enables the data stored by the integrated data repository 1104 to be further analyzed to determine trends, characteristics, features, and/or insights with respect to individuals in which one or more biological conditions may be present. For example, health insurance codes may be stored in the integrated data repository 1104 in such a way that at least one of medical procedures, biological conditions, treatments, dosages, manufacturers of medications, distributors of medications, or diagnoses may be determined for a given individual based on health insurance claims data for the individual. In various examples, the data integration and analysis system 1102 may generate and implement one or more tables that indicate correlations between health insurance claims data and various treatments, symptoms, or biological conditions that correspond to the health insurance claims data. Further, the integrated data repository 1104 may be generated using genomics data records 1506 of the group of individuals 1502. In various examples, the large amounts of health insurance claims data may be matched with genomics data for the group of individuals 1502 to generate the integrated data repository 1104.

By integrating the genomics data records 1506 for the group of individuals 1502 with the health insurance claims records 1504, the data integration and analysis system 1102 may determine correlations between the presence of one or more biomarkers that are present in the genomics data records 1506 with other characteristics of individuals that are indicated by the health insurance claims data records 1506 that existing systems are typically unable to determine. For example, the data integration and analysis system 1102 may determine one or more genomic characteristics of individuals that correspond to treatments received by individuals, timing of treatments, dosages of treatments, diagnoses of individuals, smoking status, presence of one or more biological conditions, presence of one or more symptoms of a biological condition, one or more combinations thereof, and the like. Based on the correlations determined by the data integration and analysis system 1102 using the integrated data repository 1104, cohorts of individuals that may benefit from one or more treatments may be identified that would not have been identified in existing systems. In one or more examples, the processes and techniques implemented to integrate the health insurance claims records 1504 and the genomics claims records 1506 in order to generate the integrated data repository 1104 may be complex and implement efficiency-enhancing techniques, systems, and processes in order to minimize the amount of computing resources used to generate the integrated data repository 1104.

In one or more illustrative examples, the data pipeline system 1138 may access information stored by the integrated data repository 1104 to generate datasets that include a number of additional data records 1508 that include information related to at least a portion of the group of individuals 1502. In the illustrative example of FIG. 5, the additional data record 1508 includes information indicating whether individuals are included in a cohort of individuals in which lung cancer is present. The data pipeline system 1138 may execute a plurality of different sets of data processing instructions to determine a cohort of the group of individuals 1502 in which lung cancer is present. In various examples, the additional data record 1508 may indicate information used to determine a status of an individual 1502 with respect to lung cancer, such as one or more transaction insurance identifier, one or more international classification of diseases (ICD) codes, and one or more health insurance transaction dates. In addition to including a column that indicates whether an individual 1502 is included in the lung cancer cohort, the additional data record 1508 may include a column indicating a confidence level of the status of the individual 1502 with respect to the presence of lung cancer.

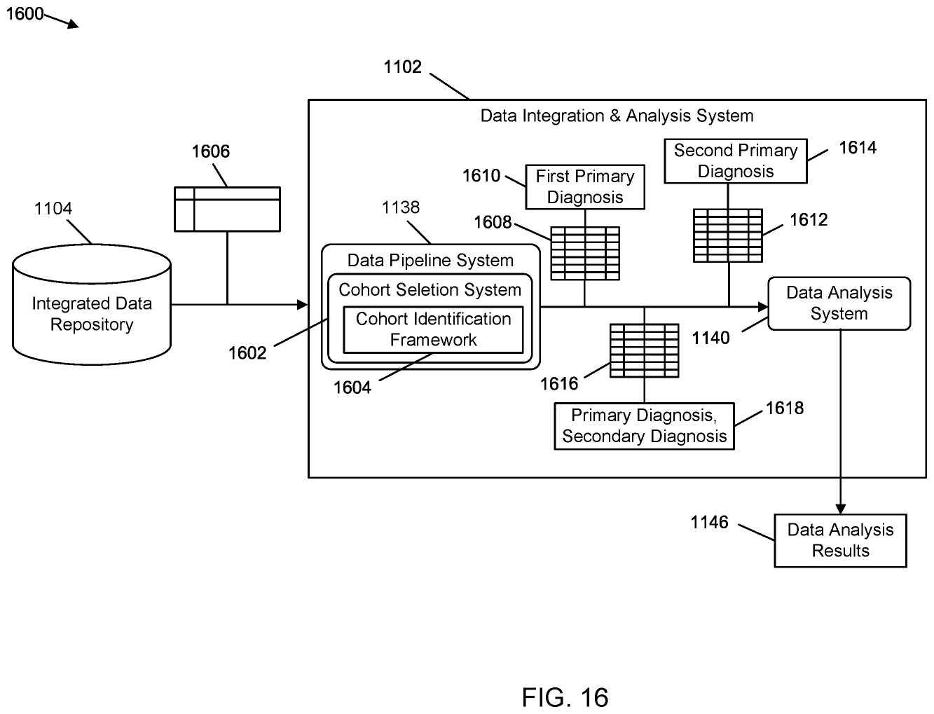

FIG. 16 illustrates a system 1600 to determine cohorts of patients having at least a primary diagnosis of a biological condition, in accordance with one or more implementations. The system 1600 can include the data integration and analysis system 1102 and the integrated data repository 1104. The data integration and analysis system 1102 can analyze information stored by the integrated data repository 1104 to determine cohorts of patients in which one or more biological conditions is present. For example, the data integration and analysis system 1102 can determine a first group of patients having data stored by the integrated data repository 1104 in which a first biological condition is present and a second group of patients having data stored by the integrated data repository 1104 in which a second biological condition is present. The data analysis and integration system 1102 can include at least the data pipeline system 1138 and the data analysis system 1140. The data analysis system 1140 can generate data analysis results 1146 based on data obtained from the data pipeline system 1138. The data analysis system 1140 can also analyze additional data obtained from the integrated data repository 1140 to generate the data analysis results 1146.

The data pipeline system 1138 can include a cohort selection system 1602 that analyzes data obtained from the integrated data repository 1104 to determine the cohorts of patients in which the one or more biological conditions are present. In various examples, the cohort selection system 1602 can analyze data from tens of thousands of patients up to hundreds of thousands of patients or more to determine cohorts of the patients. In one or more examples, hundreds of thousands of health insurance claims records up to millions of health insurance claim records or more are analyzed by the cohort selection system 1602 to determine one or more cohorts of patients. Additionally, the cohort selection system 1602 can analyze millions of insurance claims codes up to tens of millions of insurance claims codes or more to determine one or more cohorts of patients. In one or more illustrative examples, the cohort selection system 1602 can analyze information obtained from the integrated data repository 1104 according to a cohort identification framework 1604 in order to efficiently analyze such large amounts of information. The cohort identification framework 1604 can include at least one of a number of rules, a number of schema, or logic by which to analyze data obtained from the integrated data repository 1104. The cohort identification framework 1604 also provides a structure for identifying information stored within the integrated data repository 1104 that can be used to accurately identify patients in which a given biological condition is present. The cohort identification framework 1604 can be determined by implementing and/or training at least one of one or more machine learning techniques, one or more statistical techniques, or one or additional computational techniques in relation to a corpus of data related to the diagnosis of patients using health insurance data. In various illustrative examples, the cohort identification framework 1604 can include at least a portion of the processes described with respect to FIGS. 4-10.

In one or more examples, a medical headers data table 1606 can be provided to the cohort selection system 1602 to analyze according to the cohort identification framework 1604 to determine one or more cohorts of patients. The medical headers data table 1606 can include a plurality of rows and a plurality of columns. The plurality of rows can correspond to medical encounters for a number of patients. The medical encounters can correspond to visits to one or more healthcare providers, services rendered by one or more healthcare providers, therapeutics provided to one or more patients, or one or more combinations thereof. In various examples, individual medical encounters can indicate charges for at least one of service or products provided to a number of patients. For example, the individual medical encounters can indicate one or more health insurance claims related to the medical encounter. In one or more additional examples, the data table 1606 can indicate that individual patients are associated with one or more medical encounters. Individual medical encounters can correspond to a given medical encounters key and/or a given medical encounters identifier. In at least some examples, individual medical encounters keys or individual medical encounters identifiers can uniquely identify a given medical encounter. The medical headers data table 1606 can also include one or more columns that indicate dates of service for individual medical encounters. Additionally, the medical headers data table 1606 can include one or more columns that indicate diagnosis codes for patients in relation to individual medical encounters. To illustrate, the medical headers data table 1606 can indicate one or more biological conditions for which individual patients received treatment.

The cohort identification framework 1604 can indicate one or more columns of the medical headers data table 1606 to be analyzed by the cohort selection system 1602 to determine one or more cohorts of patients. To illustrate, the cohort identification framework 1604 can indicate one or more columns of the medical headers data table 1606 that include health insurance codes that correspond to the diagnosis of a biological condition. The cohort identification framework 1604 can also indicate formats of one or more types of health insurance diagnosis codes. For example, the cohort identification framework 1604 can indicated formats of international classification of disease (ICD) codes. In one or more illustrative examples, the cohort identification framework 1604 can indicate a format of ICD version 9 codes, a format of ICD version 10 codes, a format of ICD version 11 codes, or a format of another ICD version.

In addition, the cohort identification framework 1604 can indicate diagnosis codes that correspond to one or more biological conditions. In various examples, the cohort identification framework 1604 can indicate diagnosis codes that correspond to one or more forms of a given biological condition, such as one or more forms of cancer. To illustrate, the cohort identification framework 1604 can indicate one or more first diagnosis codes that correspond to a first form of cancer and one or more second diagnosis codes that correspond to a second form of cancer. In one or more additional illustrative examples, the cohort identification framework 1604 can indicate one or more first diagnosis codes of a first format that correspond to a first form of cancer, one or more second diagnosis codes of a second format that correspond to the first form of cancer, one or more third diagnosis codes of the first format that correspond to a second form of cancer, and one or more fourth diagnosis codes of the second format that correspond to the second form of cancer. In one or more examples, the diagnosis codes can correspond to a biological condition being a primary diagnosis for a patient. In one or more examples, the cohort identification framework 1604 can include diagnosis codes that indicate one or more biological conditions that do not indicate the presence of a biological condition with respect to one or more patients.

The cohort identification framework 1604 can indicate at least one of logic or rules for analyzing information included in the medical headers data table 1606. For example, the cohort identification framework 1604 can indicate threshold periods of time to be used to determine at least one of a primary diagnosis for a patient or a secondary diagnosis for a patient. In one or more illustrative examples, the cohort identification framework 1604 can indicate that a patient having a medical encounter corresponding to a first diagnosis code and another medical encounter corresponding to a second diagnosis code within a threshold period of time may be excluded from a first cohort that corresponds to a first biological condition related to the first diagnosis code. Additionally, in these scenarios, the cohort identification framework 1604 can indicate that the patient may be excluded from a second cohort that corresponds to a second biological condition related to the second diagnosis code. Further, the cohort identification framework 1604 can indicate that a patient having a medical encounter indicating a new diagnosis code more than an additional threshold period of time after a previous medical encounter indicating an initial diagnosis code may be excluded from a cohort that corresponds to a biological condition related to the initial diagnosis code. In various examples, the cohort identification framework 1604 can indicate logic for determining how to categorize a patient in situations where a patient receives treatment for multiple biological conditions on a same date.

Further, the cohort identification framework 1604 can include at least one of logic or one or more criteria to determine a quality metric with respect to a diagnosis of a patient by the cohort selection system 1602 and to include the patient in a cohort. The quality metric can correspond to a probability that a diagnosis of a patient by the cohort selection system 1602 corresponds to a biological condition that is present in the patient. In one or more examples, the quality metric can be a quantitative metric, such as a score or range of probabilities. In one or more additional examples, the quality metric can be a qualitative metric, such as “low”, “medium”, or “high”.

In one or more examples, the cohort selection system 1602 can implement the cohort identification framework 1604 to generate a first cohort data table 1608 that corresponds to a first cohort of patients having health insurance records stored by the integrated data repository 1104. The first cohort data table 1608 can correspond to a first primary diagnosis 1610 of the patients included in the first cohort. The cohort selection system 1602 can also implement the cohort identification framework 1604 to generate a second cohort data table 1612 that corresponds to a second cohort of patients having health insurance records stored by the integrated data repository 1104. The second cohort data table 1612 can correspond to a second primary diagnosis 1614 of patients included in the second cohort. Additionally, the cohort selection system 1602 can implement the cohort identification framework 1604 to generate a third cohort data table 1616 that corresponds to a third cohort of patients having health insurance records stored by the integrated data repository 1104. The third cohort data table 1616 can correspond to patients having multiple diagnoses 1618, such as a primary diagnosis and a secondary diagnosis. In one or more illustrative examples, the first primary diagnosis 1610 can include a first biological condition, such as type II diabetes, and the second primary diagnosis can include a second biological condition, such as hypertension. In one or more additional illustrative examples, the multiple diagnoses 1618 can correspond to a primary diagnosis of type II diabetes and a secondary diagnosis of hypertension. In one or more further illustrative examples, the first primary diagnosis 1610 can correspond to a first form of cancer, the second primary diagnosis 1614 can correspond to a second form of cancer, and the multiple diagnoses 1618 can correspond to patients having cancer that has metastasized, such that the patient has a primary diagnosis of the first form of cancer and a secondary diagnosis of the second form of cancer.

In various examples, at least one of the first cohort data table 1608, the second cohort data table 1612, or the third cohort data table 1616 can include information about patients included in the respective cohorts. For example, the data tables 1608, 1612, 1616 can indicate identifiers of patients having data stored by the integrated data repository 1104. The data tables 1608, 1612, 1618 can also indicate personal information of patients, such as age of patients, year of birth of patients, date of birth of patients, year of birth of patients, date of death of patients, one or more dates of health insurance claims activity, primary diagnosis of patients, secondary diagnosis of patients, metastatic status of patients, one or more combinations thereof, and so forth.

In one or more examples, the data tables 1608, 1612, 1616 can be provided to the data analysis system 1140 and the data analysis system 1140 can use at least a portion of the information stored by the data tables 1608, 1612, 1616 to generate the data analysis results 1146. In various examples, the data analysis system 1140 can use at least a patient identifier to retrieve additional data corresponding to patients included in at least one of the data tables 1608, 1612, 1616 from the integrated data repository 1104. In one or more additional examples, the data analysis system 1140 can use a patient identifier in combination with additional information, such as age of a patient, birth year of a patient, birthdate of a patient, and the like, to retrieve additional data corresponding to patients included in at least one of the data tables 1608, 1612, 1616 from the integrated data repository 1104. In one or more illustrative examples, the data analysis system 1140 can information stored by at least one of the data tables 1608, 1612, 1616 to retrieve at least one of genomics information, metabolomic information, transcriptomic information, fragmentomic information, immune receptor information, methylation information, epigenomic information, and/or proteomic information of patients to generate the data analysis results 1146.

In at least some examples, the cohort selection system 1602 can analyze data included in the medical headers data table 1606 to determine patients having a set of health insurance claims codes in one or more diagnosis columns of the medical headers data table 1604. The health insurance claims codes can correspond to a set of ICD version 9 codes and/or a set of ICD version 10 codes that correspond to a primary diagnosis of a biological condition. For example, the cohort selection system 1602 can analyze the medical headers data table 1606 to identify patients having one or more ICD version 9 diagnosis codes that correspond to non-small cell lung cancer and one or more ICD version 10 diagnosis codes that correspond to non-small cell lung cancer.

The cohort selection system 1602 can generate an intermediate data table that stores identification information of a first number of patients that correspond to the specified health insurance claims codes. In one or more illustrative examples, the intermediate data table can be temporarily stored in memory, such as in a cache memory, while additional analysis is performed by the cohort selection system 1602 with respect to data related to the first number of patients. For example, the cohort selection system 1602 can analyze additional health insurance claims data for the first number of patients according to the cohort identification framework 1604. In this way, the cohort selection system 1602 can identify patients that may have a diagnosis of a biological condition that corresponds to the specified health insurance claims codes, but the biological condition is not the primary diagnosis of the patients. In this way, the cohort selection system 1602 can implement a multi-step analysis that uses one or more intermediate data tables to accurately and efficiently determine the patients to include in the data tables 1608, 1612, 1616. In various examples, the cohort selection system 1602 can implement the cohort identification framework 1604 in relation to logic correspond to dates of diagnosis and/or in relation to additional health insurance claims codes to determine a second number of patients having a primary diagnosis of the biological condition.

In one or more further examples, the cohort selection system 1602 can analyze additional information to determine patients to include in the data tables 1608, 1612, 1616. For example, the cohort selection system 1602 can analyze histology information stored by the integrated data repository 1104 in addition to health insurance claims data to identify patients to include in a cohort having a biological condition associated with the cohort. To illustrate, histology records can also include diagnosis information. In these scenarios, the cohort selection system 1602 can analyze the diagnosis information for a biological condition stored by the integrated data repository 1104 in conjunction with health insurance claims data related to diagnosis of the biological condition to determine patients to include in a cohort. In one or more illustrative examples, the cohort selection system 1602 can analyze the histology information and the health insurance claims data according to the cohort identification framework 1604 to determine one or more patients of a cohort having a primary diagnosis related to the biological condition.

The cohort selection system 1602 can also generate one or more additional data tables. For example, the cohort selection system 1602 can generate a diagnosis data table indicating one or more diagnoses for individual patients. To illustrate, the cohort selection system 1602 can determine that patients are included in one or more cohorts. In these scenarios, the cohort selection system 1602 can indicate in the diagnosis data table that the patients are diagnosed with the biological conditions that correspond to the one or more cohorts that include the patients. The diagnosis data table can indicate biological conditions that are correspond to primary diagnoses of patients, additional biological conditions that correspond to secondary diagnoses of patients, metastatic condition of patients, or one or more combinations thereof. Also implement to determine a diagnosis table for individual patients. Indicates different diagnoses of patients over time. In addition to the diagnoses of patients, the diagnosis data table can also indicate health insurance codes that correspond to the diagnoses, identifiers of patients, treatment dates, dates related to diagnosis of the patients, a most recent diagnosis, or one or more combinations thereof. In various examples, the data analysis system 1140 can analyze information included in the diagnosis data table or information derived from the diagnosis data table to generate the data analysis results 1146. In one or more illustrative examples, the diagnosis data table can be used to generate real world evidence metrics, such as real world overall survival (rwOS), to determine the data analysis results 1146.

In one or more illustrative examples, the data analysis system 1140 may analyze information stored by one or more of the data tables 1608, 1612, 1616 and/or information retrieved from the integrated data repository 1104 based on one or more of the data tables 1608, 1612, 1616 to determine the data analysis results 1146. In one or more examples, the data analysis system 1140 may receive a request to analyze information that corresponds to a cohort of patients treated for a given biological condition. In response to the request, the data analysis system 1140 may analyze information generated by the cohort selection system 1602 to generate data analysis results 1146 that include one or more quantitative measures corresponding to patients included in one or more cohorts. To illustrate, the data analysis system 1140 may analyze information generated by the cohort selection system 1602 to determine real world survival metrics for patients included in a cohort. In various examples, the data analysis system 1140 may analyze information related to a cohort of patients to determine a survival probability over a period of time for patients included in the cohort. In one or more illustrative examples, the data analysis system 1140 may information related to one or more cohorts of patients to determine real-world overall survival metrics for the patients included in the cohorts. In one or more additional illustrative examples, the data analysis system 1140 may analyze information related to cohorts identified by the cohort selection system 1602 to determine time-to-next-treatment metrics and/or time to discontinuation metrics for patients included in one or more cohorts.

In various examples, the data analysis system 1140 may analyze information that correspond to patients included in a cohort identified by the cohort selection system 1602 to determine an amount of progression of the biological condition within at least a subset of the patients included in cohort. In one or more examples, the data analysis system 1140 may determine an amount of progression for a cohort of patients receiving one or more pharmaceutical substances as part of a line of therapy based on an analysis of information generated by the cohort selection system 1602. Additionally, the data analysis system 1140 may determine an amount of progression for a cohort of patients having one or more genomic mutations based on an analysis of information generated by the cohort selection system 1602. In one or more illustrative examples, the data analysis system 1140 may analyze at least one of time-to-next-treatment metrics or time to discontinuation metrics for a cohort of patients to determine an amount of progression of the biological condition for patients of the cohort having the genomic mutations. In these instances, the data analysis system 1140 may query the integrated data repository 1104 to determine genomic data of patients included in the cohort and identify patients of the cohort having one or more specified genomic mutations. The data analysis system 1140 may then analyze time-to-next-treatment metrics, time to discontinuation metrics, and/or real-world overall survival metrics of patients included in the cohort having the one or more genomic mutations to determine progression of a biological condition for patients included in the cohort and that received the treatment for the biological condition.

In one or more further examples, the data analysis system 1140 may analyze information generated by the cohort selection system 1602 to determine a level of resistance developed by one or more patients included in a cohort receiving one or more treatments for the biological condition associated with the cohort. For example, the data analysis system 1140 may analyze information of cohorts of patients identified by the cohort selection system 1602 to determine a level of resistance in one or more patients of the cohort that received one or more pharmaceutical substances as part of a line of therapy to treat the biological condition of patients included in the cohort. In various examples, the data analysis system 1140 may analyze at least one of time-to-next-treatment metrics, time to discontinuation metrics, or real-world survival metrics to determine a level of resistance developed by patients of the cohort that received treatment. In at least some examples, the data analysis system 1140 may also determine a level of resistance with respect to one or more treatments for patients in the cohort having one or more genomic mutations. In at least some examples, the level of resistance may be greater in situations where a time-to-next-treatment or a real world survival rate have lower values and the level of resistance may be lower in situations where values of time-to-next-treatment or real-world survival rate are relatively higher.

In at least some examples, the data analysis system 108 may analyze lines of therapy information stored by the one or more lines of therapy data structures 836 that correspond to a biological condition to determine a recommendation for one or more treatments to administer to a patient diagnosed with a biological condition. In one or more examples, the data analysis system 1140 may information about cohorts of patients identified by the cohort selection system 1602 to determine one or more characteristics of patients of the cohort that received one or more lines of therapy in which a level of resistance is relatively low and/or an amount of progression is relatively low. The data analysis system 1140 may then analyze characteristics of one or more additional patients of the cohort diagnosed with the biological condition to determine whether to recommend the one or more lines of therapy as treatment to the one or more additional patients. At least a portion of the one or more additional patients of the cohort may have already received treatment for the biological condition. In one or more additional examples, at least a portion of the one or more additional patients of the cohort may not have received treatment for the biological condition associated with the cohort. In various examples, the data analysis system 1140 may also analyze information of patients included in a given cohort to determine an effectiveness of a line of therapy for the patients included in the cohort. The effectiveness of the line of therapy may correspond to a probability of the line of therapy at least one of reducing the effects of or eliminating the biological condition with respect the patients of the cohort.

In various examples, an amount of progression of the biological condition, an effectiveness of a line of therapy to treat the biological condition, the probability of developing resistance to a line of treatment, or a combination thereof, may be determined by the data analysis system 1140 using at least one of one or more statistical techniques or one or more machine learning techniques. To illustrate, the data analysis system 1140 may implement at least one of Cox proportional hazards models, chi-squared tests, log-rank tests, or Kaplan-Meier methods to determine at least one of an amount of progression of the biological condition, an effectiveness of a line of therapy to treat the biological condition, or the probability of developing resistance to a line of treatment. In one or more additional examples, the data analysis system 1140 may implement one or more neural networks, one or more convolutional neural networks, or one or more residual neural networks to determine at least one of an amount of progression of the biological condition, an effectiveness of a line of therapy to treat the biological condition, or the probability of developing resistance to a line of treatment.

In one or more illustrative examples, the data analysis system 1140 may determine one or more characteristics of patients that have at least one of less than a threshold probability of developing resistance to a line of therapy or at least at an additional threshold amount of effectiveness for the line of therapy. In one or more scenarios, the data analysis system 1140 may analyze information about cohorts of patients determined by the cohort selection system 1602 to determine the one or more characteristics. In at least some examples, the data analysis system 1140 may implement at least one of one or more statistical techniques or one or more machine learning techniques to determine the one or more characteristics of patients that have at least one of less than a threshold probability of developing resistance to a line of therapy or at least at an additional threshold amount of effectiveness for the line of therapy. In one or more examples, the data analysis system 1140 may implement at least one of one or more extraction algorithms or one or more classification algorithms to determine the one or more characteristics. In various examples, the data analysis system 1140 may implement at least one of one or more neural networks, one or more feedforward neural networks, one or more recurrent neural networks, one or more residual networks, or one or more autoencoders to determine the one or more characteristics that have at least one of less than a threshold probability of developing resistance to a line of therapy or at least at an additional threshold amount of effectiveness for the line of therapy.

In one or more additional illustrative examples, the data analysis system 1140 may implement one or more log-rank tests to analyze differences between time to death metrics and time-to-next-treatment metrics determined based on information of one or more cohorts of patients determined by the cohort selection system 1602 having one or more genomic mutations and diagnosed with a given biological condition or in which the given biological condition is suspected to be present. In various examples, the patients included in the analysis may also have received one or more specified lines of therapy to treat the biological condition. Additionally, the data analysis system 1140 may implement one or more Chi-squared tests to determine the proportion of patients included in a cohort, having one or more specified genomic mutations and, in at least some instances, one or more co-occurring genomic mutations in patients of the cohort having one or more additional genomic characteristics, such as one or more clonal genomic mutations versus one or more sub-clonal genomic mutations. Further, one or more Cox proportional hazards models may be implemented by the data analysis system 1140 to determine survival metrics for the patients. In this way, the effectiveness of one or more lines of therapy to treat the biological condition may be determined by the data analysis system 1140 based on survival probabilities determined using the Cox proportional hazards models.

The cohort identification framework 1604 in addition to one or more computational techniques implemented by the cohort selection system 1602 and, in at least some instances, the intermediate data tables generated by the cohort selection system 1602 may be used by the data analysis system 1140 to accurately generate the data analysis results 1146. That is, based on the cohorts identified by the cohort selection system 1602, real world survival metrics, disease progression metrics, disease resistance metrics, treatment effectiveness levels, one or more combinations thereof, and so forth may be accurately determined for the cohort because the patients included in the cohort have at least a threshold probability of a given biological condition being present. The accurate determination of these quantitative measures enables the data analysis system 1140 to provide treatment recommendations to patients that are accurate, effective, and result in improved outcomes for patients. Without the procedures, rules, schema, and protocols specified in the cohort identification framework 1604 and the computational techniques implemented by the cohort selection system 1602 and the data analysis system 1140, the treatment recommendations included in the data analysis results 1146 are not as likely to improve outcomes for patients. The cohort identification framework 1604 has been generated over time using a number of computational techniques, training processes, and feedback loops to determine a specified set of criteria, rules, schema, protocols, thresholds, and computational techniques that produce optimal treatment recommendations, provide accurate metrics indicating the effectiveness of lines of therapy on outcomes for cohorts of patients, and provide accurate information regarding the impact of genomic mutations of patient cohorts on treatment outcomes.

FIG. 17 illustrates a circuit block diagram of a computing machine 1700 in accordance with some implementations. In some implementations, components of the computing machine 1700 may store or be integrated into other components shown in the circuit block diagram of FIG. 17. For example, portions of the computing machine 1700 may reside in the processor 1702 and may be referred to as “processing circuitry.” Processing circuitry may include processing hardware, for example, one or more central processing units (CPUs), one or more graphics processing units (GPUs), and the like. In alternative implementations, the computing machine 1700 may operate as a standalone device or may be connected (e.g., networked) to other computers. In a networked deployment, the computing machine 1700 may operate in the capacity of a server, a client, or both in server-client network environments. In an example, the computing machine 1700 may act as a peer machine in peer-to-peer (P2P) (or other distributed) network environment. In this document, the phrases P2P, device-to-device (D2D) and sidelink may be used interchangeably. The computing machine 1700 may be a specialized computer, a personal computer (PC), a tablet PC, a personal digital assistant (PDA), a mobile telephone, a smart phone, a web appliance, a network router, switch or bridge, or any machine capable of executing instructions (sequential or otherwise) that specify actions to be taken by that machine.

Examples, as described herein, may include, or may operate on, logic or a number of components, modules, or mechanisms. Modules and components are tangible entities (e.g., hardware) capable of performing specified operations and may be configured or arranged in a certain manner. In an example, circuits may be arranged (e.g., internally or with respect to external entities such as other circuits) in a specified manner as a module. In an example, the whole or part of one or more computer systems/apparatus (e.g., a standalone, client or server computer system) or one or more hardware processors may be configured by firmware or software (e.g., instructions, an application portion, or an application) as a module that operates to perform specified operations. In an example, the software may reside on a machine readable medium. In an example, the software, when executed by the underlying hardware of the module, causes the hardware to perform the specified operations.

Accordingly, the term “module” (and “component”) is understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein. Considering examples in which modules are temporarily configured, each of the modules need not be instantiated at any one moment in time. For example, where the modules comprise a general-purpose hardware processor configured using software, the general-purpose hardware processor may be configured as respective different modules at different times. Software may accordingly configure a hardware processor, for example, to constitute a particular module at one instance of time and to constitute a different module at a different instance of time.

The computing machine 1700 may include a hardware processor 1702 (e.g., a central processing unit (CPU), a GPU, a hardware processor core, or any combination thereof), a main memory 1704 and a static memory 1706, some or all of which may communicate with each other via an interlink (e.g., bus) 1708. Although not shown, the main memory 1704 may contain any or all of removable storage and non-removable storage, volatile memory or non-volatile memory. The computing machine 1700 may further include a video display unit 1710 (or other display unit), an alphanumeric input device 1712 (e.g., a keyboard), and a user interface (UI) navigation device 1714 (e.g., a mouse). In an example, the display unit 1710, input device 1712 and UI navigation device 1714 may be a touch screen display. The computing machine 1700 may additionally include a storage device (e.g., drive unit) 1716, a signal generation device 1718 (e.g., a speaker), a network interface device 1720, and one or more sensors 1621, such as a global positioning system (GPS) sensor, compass, accelerometer, or other sensor. The computing machine 1700 may include an output controller 1728, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The drive unit 1716 (e.g., a storage device) may include a machine readable medium 1722 on which is stored one or more sets of data structures or instructions 1724 (e.g., software) embodying or utilized by any one or more of the techniques or functions described herein. The instructions 1724 may also reside, completely or at least partially, within the main memory 1704, within static memory 1706, or within the hardware processor 1702 during execution thereof by the computing machine 1700. In an example, one or any combination of the hardware processor 1702, the main memory 1704, the static memory 1706, or the storage device 1716 may constitute machine readable media.

While the machine readable medium 1722 is illustrated as a single medium, the term “machine readable medium” may include a single medium or multiple media (e.g., a centralized or distributed database, and/or associated caches and servers) configured to store the one or more instructions 1724.

The term “machine readable medium” may include any medium that is capable of storing, encoding, or carrying instructions for execution by the computing machine 1700 and that cause the computing machine 1700 to perform any one or more of the techniques of the present disclosure, or that is capable of storing, encoding or carrying data structures used by or associated with such instructions. Non-limiting machine readable medium examples may include solid-state memories, and optical and magnetic media. Specific examples of machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; Random Access Memory (RAM); and CD-ROM and DVD-ROM disks. In some examples, machine readable media may include non-transitory machine readable media. In some examples, machine readable media may include machine readable media that is not a transitory propagating signal.

The instructions 1724 may further be transmitted or received over a communications network 1726 using a transmission medium via the network interface device 1720 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, a Long Term Evolution (LTE) family of standards, a Universal Mobile Telecommunications System (UMTS) family of standards, peer-to-peer (P2P) networks, among others. In an example, the network interface device 1720 may include one or more physical jacks (e.g., Ethernet, coaxial, or phone jacks) or one or more antennas to connect to the communications network 1726.

Some implementations are described as numbered examples (Example 1, 2, 3, etc.). These are provided as examples only and do not limit the technology disclosed herein.

Example 1 is a method implemented at one or more computing machines comprising processing circuitry and memory, the method comprising: accessing, at the processing circuitry, one or more medical data repositories storing data for a given patient from among a plurality of patients, the one or more medical data repositories storing pharmacy data, medical office visit data, and medical insurance transaction data; identifying, based on one or more disease codes in the medical office visit data or the medical insurance transaction data, one or more biological conditions and a metastatic state for the given patient; identifying, based on one or more drug codes in the pharmacy data, one or more lines of treatment for the given patient; identifying, based on one or more insurance codes in the medical insurance transaction data, one or more medical procedures undergone by the given patient; determining, based on a combination of the one or more biological conditions, the metastatic state, the one or more lines of treatment, and the one or more medical procedures, a primary diagnosis biological condition for the given patient; assigning, based on the primary diagnosis biological condition, the given patient to a cohort of patients; and providing an output representing the assigned cohort for the given patient.

In Example 2, the subject matter of Example 1 includes, wherein determining the primary diagnosis biological condition comprises: creating, based on the medical insurance transaction data, a master gap table indicating intervals between two successive procedures for the given patient that have a same treatment name and insurance code, wherein the master gap table comprises columns for treatment name, insurance code, units, and gap length; computing, based on the master gap table, a median gap table indicating the median gap for each combination of treatment name and insurance code, wherein the median gap table comprises columns for treatment name, insurance code, units, and gap length; and determining the primary diagnosis biological condition based, at least in part, on data in the median gap table.

In Example 3, the subject matter of Examples 1-2 includes, wherein the processing circuitry comprises a plurality of multithreaded graphics processing units (GPUs), the method further comprising: determining, in parallel and using parallel threads of the plurality of multithreaded GPUs, assigned cohorts for multiple patients, including the given patient, from the plurality of patients.

In Example 4, the subject matter of Examples 1-3 includes, wherein: the disease codes comprise International Classification of Diseases (ICD) codes, the drug codes comprise National Drug Code (NDC) codes, and the insurance codes comprise Healthcare Common Procedure Coding System (HCPCS) codes.

In Example 5, the subject matter of Example 4 includes, wherein: identifying, based on the one or more disease codes in the medical office visit data or the medical insurance transaction data, the one or more biological conditions for the given patient comprise: identifying that the given patient has lung cancer based on ICD codes associated with lung cancer.

In Example 6, the subject matter of Example 5 includes, wherein: identifying, based on the one or more disease codes in the medical office visit data or the medical insurance transaction data, the metastatic state for the given patient is based on a secondary malignancy ICD code or HCPCS code.

In Example 7, the subject matter of Examples 1-6 includes, wherein the primary diagnosis biological condition for the given patient is determined based on disease codes, drug codes or insurance codes associated with a date within a predefined date range.

In Example 8, the subject matter of Examples 1-7 includes, wherein assigning the given patient to the cohort comprises: analyzing one or more data tables that include medical insurance transaction data for the given patient, the one or more data tables being from the one or more medical data repositories; determining one or more first insurance transactions that include one or more first code identifiers included in the one or more data tables, wherein the one or more first code identifiers correspond to diagnoses of the patient with respect to the one or more biological conditions; determining one or more second insurance transactions that include one or more second code identifiers included in the one or more data tables, wherein the one or more second code identifiers corresponding to medical procedures administered with respect to the patient, the medical procedures being from the medical office visit data; generating a medical headers table that includes a first number of columns storing the one or more first code identifiers, a second number of columns storing the one or more second code identifiers, and a plurality of rows with individual rows of the plurality of rows corresponding to a first medical insurance transaction of the one or more first insurance transactions or a second medical insurance transaction of the one or more second insurance transactions; storing the medical headers table in the one or more medical data repositories; and determining the cohort for the patient based on data in the medical headers table.

In Example 9, the subject matter of Example 8 includes, wherein: individual first insurance transactions of the one or more first insurance transactions indicate a date of service of the individual first insurance transactions; and individual second insurance transactions of the one or more second insurance transactions indicate a date of service of the individual second insurance transactions.

In Example 10, the subject matter of Example 9 includes, arranging the plurality of rows of the medical headers table in ascending order based on the dates of service such that a claim with an earliest date of service is a first row of the medical headers table and that a transaction with a most recent date of service is a last row of the medical headers table.

In Example 11, the subject matter of Examples 8-10 includes, analyzing the one or more first code identifiers to determine that a first code identifier of the one or more first code identifiers is included in a group of insurance code identifiers that correspond to the one or more biological conditions.

In Example 12, the subject matter of Example 11 includes, wherein: the first code identifier is arranged according to a first format that corresponds to a first classification of insurance code identifiers; the first classification of insurance code identifiers corresponds to International Classification of Diseases version 9 (ICD-9).

In Example 13, the subject matter of Examples 11-12 includes, wherein: the first code identifier is arranged according to a second format that corresponds to a second classification of insurance code identifiers; the second classification of insurance code identifiers corresponds to International Classification of Diseases version 10 (ICD-10).

In Example 14, the subject matter of Examples 11-13 includes, wherein: the one or more biological conditions include a plurality of subtypes; individual subtypes of the plurality of subtypes correspond to a subset of the group of insurance code identifiers that correspond to the one or more biological conditions; and the method further comprises determining that the first code identifier is included in a first subset of the group of insurance codes that corresponds to a first subtype of the biological condition.

In Example 15, the subject matter of Examples 11-14 includes, wherein the biological condition is cancer and the plurality of subtypes include at least one of lung cancer, breast cancer, or colorectal cancer.

In Example 16, the subject matter of Example 15 includes, determining one or more third insurance transactions having a date of service that is within a predefined time period ending on a first date of service; analyzing one or more third insurance code identifiers of the third insurance transactions with respect to the first code identifiers and the second code identifiers.

In Example 17, the subject matter of Example 16 includes, determining that the one or more third insurance code identifiers are not included in the first code identifiers and the second code identifiers; and determining, based on the third insurance code identifiers, that the patient is included in a cohort of patients in which a given subtype of the one or more biological conditions is present.

In Example 18, the subject matter of Examples 16-17 includes, wherein the one or more third insurance code identifiers correspond to an additional biological condition.

In Example 19, the subject matter of Examples 16-18 includes, determining that the one or more third insurance code identifiers are included in a portion of the group of insurance code identifiers that are not included in the subset of the group of insurance code identifiers; determining that a date of service of at least one of the one or more third insurance transactions is a same date as the date of service as one of the one or more first insurance transactions; determining that there are no other additional insurance transactions having insurance code identifiers included in the group of code identifiers; and determining that the patient is included in a cohort of patients in which the subtype of the biological condition is present.

In Example 20, the subject matter of Examples 16-19 includes, determining that the one or more third insurance code identifiers are included in a portion of the group of insurance code identifiers that are not included in the subset of the group of insurance code identifiers; determining that a date service of at least one of the one or more third insurance Example is prior to the date of service of one of the one or more first insurance transactions and within the predefined time period; and determining that the patient is not included in a cohort of patients in which the subtype of the biological condition is present.

Example 21 is a method implemented at one or more computing machines comprising processing circuitry and memory, the method comprising: accessing, at the processing circuitry, one or more medical data tables storing medical insurance transaction data for a plurality of patients, the one or more medical data tables comprising a date column and a diagnosis column; identifying, using the processing circuitry and based on the diagnosis column, a set of patients having a specified biological condition, the set of patients being from among the plurality of patients; determining, for each patient in the set of patients, an earliest date when the patient received a diagnosis of the specified biological condition; identifying, using the processing circuitry and based on the diagnosis column and the date column, a cohort of patients from among the set of patients, the cohort of patients lacking a diagnosis from a collection of biological conditions associated with a date occurring during a predefined time window before the earliest date when the patient received the diagnosis of the specified biological condition; and providing an output representing the cohort.

In Example 22, the subject matter of Example 21 includes, wherein the diagnosis column stores International Classification of Diseases version 9 (ICD-9) or International Classification of Diseases version 10 (ICD-10) codes.

In Example 23, the subject matter of Examples 21-22 includes, wherein the specified biological condition is lung cancer, wherein the collection of biological conditions comprises cancers different from lung cancer, wherein the predefined time window before the earliest date is six months before the earliest date.

In Example 24, the subject matter of Examples 21-23 includes, wherein the specified biological condition is a specified type of cancer, the method further comprising: determining a metastatic state of at least one patient from the cohort.

In Example 25, the subject matter of Example 24 includes, wherein the metastatic state is determined based on a secondary malignancy International Classification of Diseases (ICD) code or Healthcare Common Procedure Coding System (HCPCS) code.

In Example 26, the subject matter of Examples 21-25 includes, wherein identifying the cohort comprises: arranging rows associated with patients in the set by date; and accessing rows associated with the predefined time window to identify patients in the set that lack the diagnosis from the collection of biological conditions during the predefined time window.

Example 27 is at least one machine-readable medium including instructions that, when executed by processing circuitry, cause the processing circuitry to perform operations to implement of any of Examples 1-26.

Example 28 is an apparatus comprising means to implement of any of Examples 1-26.

Example 29 is a system to implement of any of Examples 1-26.

Example 30 is a method to implement of any of Examples 1-26.

Example 31. A method comprising: obtaining, by a computing system having one or more processors and memory, health insurance claims data for a number of patients, the health insurance claims data indicating a number of health insurance codes for the number of patients; analyzing, by the computing system, the health insurance claims data to determine a cohort of patients of the number of patients having a primary diagnosis that corresponds to a biological condition; determine, by the computing system, a number of identifiers of individual patients included in the cohort, the number of identifiers uniquely identifying the individual patients within an integrated data repository, the integrated data repository storing health insurance claims data in conjunction with genomics data for the number of patients; analyzing, by the computing system, the genomics data for the cohort of patients to determine one or more real-world evidence metrics for individual patients included in the cohort of patients, the one or more real-world metrics indicating an amount of progression of the biological condition with respect to the individual patients included in the cohort; and analyzing, by the computing system, the one or more real-world metrics in conjunction with the genomics data for the cohort of patients to determine one or more genomic mutations that correspond to the amount of progression of the biological condition with respect to the individual patients included in the cohort.

Example 32. The method of example 31, wherein the real-world evidence metrics include at least one of a period of time between one or more treatments for the biological condition received by one or more first patients included in the cohort of patients and a date of death of the one or more first patients, a period of time between one or more first treatments received by one or more second patients included in the cohort of patients and one or more second treatments received by the one or more second patients, or a period of time between one or more treatments received by one or more third patients included in the cohort of patients and a date of a last treatment received by the one or more third patients.

Example 33. The method of example 31 or 32, wherein the health insurance codes include diagnosis codes that correspond to a number of biological conditions.

Example 34. The method of any one of examples 31-33, wherein the health insurance codes are stored in a data table that includes a number of rows that correspond to medical encounters of the number of patients, individual medical encounters including a number of health insurance codes that correspond to at least one of medical services, medical procedures, or therapeutics provided to individual patients of the number of patients in relation to treatment for one or more biological conditions.

Example 35. The method of any one of examples 31-34, comprising: determining, by the computing system, one or more candidate treatments for one or more patients included in the cohort based on at least one of the amount of progression of the biological condition with respect to individual patients of the one or more patients or the one or more genomic mutations present in the one or more patients.

Example 36. The method of any one of examples 31-35, comprising: determining, by the computing system, the cohort of patients by analyzing the health insurance claims data according to a cohort identification framework, the cohort identification framework indicating at least one of one or more rules, one or more schema, or logic to be applied to determining one or more patients to include in the cohort of patients.

Example 37. The method of example 36, wherein the cohort identification framework indicates one or more first health insurance diagnosis codes that correspond to a first biological condition and one or more second health insurance diagnosis codes that correspond to a second biological condition, and the method comprises: analyzing, by the computing system, the health insurance claims data to determine patients having health insurance claims records that include the one or more first health insurance diagnosis codes to include in the cohort of patients.

Example 38. The method of example 37, comprising: determining, by the computing system, that one or more additional health insurance diagnosis codes that correspond to one or more additional biological conditions are not present in health insurance claims data within a threshold period of time after a date of initial health insurance claims data, wherein the initial health insurance claims data included the one or more first health insurance diagnosis codes; determining, by the computing system, that the one or more patients have a primary diagnosis that corresponds to the biological condition; and determining, by the computing system, that the one or more patients are included in the cohort of patients.

Example 39. The method of example 37, comprising: determining, by the computing system, that one or more additional health insurance diagnosis codes that correspond to one or more additional biological conditions are present in health insurance claims data within a threshold period of time after a date of initial health insurance claims data, wherein the initial health insurance claims data included the one or more first health insurance diagnosis codes; determining, by the computing system, that one or more additional patients have a primary diagnosis that corresponds to an additional biological condition that does not include the biological condition; and determining, by the computing system, that the one or more additional patients are to be excluded from the cohort of patients.

Example 40. The method of example 39, wherein the biological condition is a first form of cancer and the additional biological condition is a second form of cancer, and the method comprises: determining, by the computing system, that the one or more additional patients have cancer that has metastasized.

Example 41. The method of any one of examples 31-40, comprising: generating, by the computing system, a diagnosis data table that includes a plurality of rows with individual rows corresponding to individual patients of the number of patients and the individual rows indicating one or more diagnoses of biological conditions with respect to the individual patients related to the individual rows.

Example 42. A system comprising: one or more hardware processors; and memory storing computer-readable instructions that, when executed by the one or more hardware processors, perform operations comprising: obtaining health insurance claims data for a number of patients, the health insurance claims data indicating a number of health insurance codes for the number of patients; analyzing the health insurance claims data to determine a cohort of patients of the number of patients having a primary diagnosis that corresponds to a biological condition; determine a number of identifiers of individual patients included in the cohort, the number of identifiers uniquely identifying the individual patients within an integrated data repository, the integrated data repository storing health insurance claims data in conjunction with genomics data for the number of patients; analyzing the genomics data for the cohort of patients to determine one or more real-world evidence metrics for individual patients included in the cohort of patients, the one or more real-world metrics indicating an amount of progression of the biological condition with respect to the individual patients included in the cohort; and analyzing the one or more real-world metrics in conjunction with the genomics data for the cohort of patients to determine one or more genomic mutations that correspond to the amount of progression of the biological condition with respect to the individual patients included in the cohort.

Example 43. The system of example 42, wherein the real-world evidence metrics include at least one of a period of time between one or more treatments for the biological condition received by one or more first patients included in the cohort of patients and a date of death of the one or more first patients, a period of time between one or more first treatments received by one or more second patients included in the cohort of patients and one or more second treatments received by the one or more second patients, or a period of time between one or more treatments received by one or more third patients included in the cohort of patients and a date of a last treatment received by the one or more third patients.

Example 44. The system of example 42 or 43, wherein the health insurance codes include diagnosis codes that correspond to a number of biological conditions.

Example 45. The system of any one of examples 42-44, wherein the health insurance codes are stored in a data table that includes a number of rows that correspond to medical encounters of the number of patients, individual medical encounters including a number of health insurance codes that correspond to at least one of medical services, medical procedures, or therapeutics provided to individual patients of the number of patients in relation to treatment for one or more biological conditions.

Example 46. The system of any one of examples 42-45, wherein the memory stores additional computer-readable instructions, that when executed by the one or more hardware processors, perform additional operations comprising: determining one or more candidate treatments for one or more patients included in the cohort based on at least one of the amount of progression of the biological condition with respect to individual patients of the one or more patients or the one or more genomic mutations present in the one or more patients.

Example 47. The system of any one of examples 42-46, wherein the memory stores additional computer-readable instructions, that when executed by the one or more hardware processors, perform additional operations comprising: determining the cohort of patients by analyzing the health insurance claims data according to a cohort identification framework, the cohort identification framework indicating at least one of one or more rules, one or more schema, or logic to be applied to determining one or more patients to include in the cohort of patients.

Example 48. The system of example 47, wherein the cohort identification framework indicates one or more first health insurance diagnosis codes that correspond to a first biological condition and one or more second health insurance diagnosis codes that correspond to a second biological condition, and wherein the memory stores additional computer-readable instructions, that when executed by the one or more hardware processors, perform additional operations comprising: analyzing the health insurance claims data to determine patients having health insurance claims records that include the one or more first health insurance diagnosis codes to include in the cohort of patients.

Example 49. The system of example 48, wherein the memory stores additional computer-readable instructions, that when executed by the one or more hardware processors, perform additional operations comprising: determining that one or more additional health insurance diagnosis codes that correspond to one or more additional biological conditions are not present in health insurance claims data within a threshold period of time after a date of initial health insurance claims data, wherein the initial health insurance claims data included the one or more first health insurance diagnosis codes; determining that the one or more patients have a primary diagnosis that corresponds to the biological condition; and determining that the one or more patients are included in the cohort of patients.

Example 50. The system of example 48, wherein the memory stores additional computer-readable instructions, that when executed by the one or more hardware processors, perform additional operations comprising: determining that one or more additional health insurance diagnosis codes that correspond to one or more additional biological conditions are present in health insurance claims data within a threshold period of time after a date of initial health insurance claims data, wherein the initial health insurance claims data included the one or more first health insurance diagnosis codes; determining that one or more additional patients have a primary diagnosis that corresponds to an additional biological condition that does not include the biological condition; and determining that the one or more additional patients are to be excluded from the cohort of patients.

Example 51. The system of example 50, wherein the biological condition is a first form of cancer and the additional biological condition is a second form of cancer, and wherein the memory stores additional computer-readable instructions, that when executed by the one or more hardware processors, perform additional operations comprising: determining that the one or more additional patients have cancer that has metastasized.

Example 52. The system of any one of examples 42-51, wherein the memory stores additional computer-readable instructions, that when executed by the one or more hardware processors, perform additional operations comprising: generating a diagnosis data table that includes a plurality of rows with individual rows corresponding to individual patients of the number of patients and the individual rows indicating one or more diagnoses of biological conditions with respect to the individual patients related to the individual rows.

As used herein, a component, can refer to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example implementations, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

It should be understood that the individual steps used in the methods of the present teachings may be performed in any order and/or simultaneously, as long as the teaching remains operable. Furthermore, it should be understood that the apparatus and methods of the present teachings can include any number, or all, of the described implementations, as long as the teaching remains operable.

The various steps of the methods disclosed herein, or the steps carried out by the systems disclosed herein, may be carried out at the same time or different times, and/or in the same geographical location or different geographical locations, e.g., countries. The various steps of the methods disclosed herein can be performed by the same person or different people.

Various implementations of systems, devices, and methods have been described herein. These implementations are given only by way of example and are not intended to limit the scope of the claimed inventions. It should be appreciated, moreover, that the various features of the implementations that have been described may be combined in various ways to produce numerous additional implementations. Moreover, while various materials, dimensions, shapes, configurations and locations, etc. have been described for use with disclosed implementations, others besides those disclosed may be utilized without exceeding the scope of the claimed inventions.

Persons of ordinary skill in the relevant arts will recognize that implementations may comprise fewer features than illustrated in any individual implementation described above. The implementations described herein are not meant to be an exhaustive presentation of the ways in which the various features may be combined. Accordingly, the implementations are not mutually exclusive combinations of features; rather, implementations can comprise a combination of different individual features selected from different individual implementations, as understood by persons of ordinary skill in the art. Moreover, elements described with respect to one implementation can be implemented in other implementations even when not described in such implementations unless otherwise noted. Although a dependent claim may refer in the claims to a specific combination with one or more other claims, other implementations can also include a combination of the dependent claim with the subject matter of each other dependent claim or a combination of one or more features with other dependent or independent claims. Such combinations are proposed herein unless it is stated that a specific combination is not intended. Furthermore, it is intended also to include features of a claim in any other independent claim even if this claim is not directly made dependent to the independent claim.

Moreover, reference in the specification to “one implementation,” “an implementation,” or “some implementations” means that a particular feature, structure, or characteristic, described in connection with the implementation, is included in at least one implementation of the teaching. The appearances of the phrase “in one implementation” in various places in the specification are not necessarily all referring to the same implementation.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

Although an implementation has been described with reference to specific example implementations, it will be evident that various modifications and changes may be made to these implementations without departing from the broader spirit and scope of the present disclosure. Accordingly, the specification and drawings are to be regarded in an illustrative rather than a restrictive sense. The accompanying drawings that form a part hereof show, by way of illustration, and not of limitation, specific implementations in which the subject matter may be practiced. The implementations illustrated are described in sufficient detail to enable those skilled in the art to practice the teachings disclosed herein. Other implementations may be utilized and derived therefrom, such that structural and logical substitutions and changes may be made without departing from the scope of this disclosure. This Detailed Description, therefore, is not to be taken in a limiting sense, and the scope of various implementations is defined only by the appended claims, along with the full range of equivalents to which such claims are entitled.

Although specific implementations have been illustrated and described herein, it should be appreciated that any arrangement calculated to achieve the same purpose may be substituted for the specific implementations shown. This disclosure is intended to cover any and all adaptations or variations of various implementations. Combinations of the above implementations, and other implementations not specifically described herein, will be apparent to those of skill in the art upon reviewing the above description.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, user equipment (UE), article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

1. A method implemented at one or more computing machines comprising processing circuitry and memory, the method comprising:
accessing, at the processing circuitry, one or more medical data repositories storing data for a given patient from among a plurality of patients, the one or more medical data repositories storing pharmacy data, medical office visit data, and medical insurance transaction data;
identifying, based on one or more disease codes in the medical office visit data or the medical insurance transaction data, one or more biological conditions and a metastatic state for the given patient;
identifying, based on one or more drug codes in the pharmacy data, one or more lines of treatment for the given patient;
identifying, based on one or more insurance codes in the medical insurance transaction data, one or more medical procedures undergone by the given patient;
determining, based on a combination of the one or more biological conditions, the metastatic state, the one or more lines of treatment, and the one or more medical procedures, a primary diagnosis biological condition for the given patient;
assigning, based on the primary diagnosis biological condition, the given patient to a cohort of patients; and
providing an output representing the assigned cohort for the given patient.

2. The method of claim 1, wherein determining the primary diagnosis biological condition comprises:
creating, based on the medical insurance transaction data, a master gap table indicating intervals between two successive procedures for the given patient that have a same treatment name and insurance code, wherein the master gap table comprises columns for treatment name, insurance code, units, and gap length;
computing, based on the master gap table, a median gap table indicating the median gap for each combination of treatment name and insurance code, wherein the median gap table comprises columns for treatment name, insurance code, units, and gap length; and
determining the primary diagnosis biological condition based, at least in part, on data in the median gap table.

3. The method of claim 1, wherein the processing circuitry comprises a plurality of multithreaded graphics processing units (GPUs), the method further comprising:
determining, in parallel and using parallel threads of the plurality of multithreaded GPUs, assigned cohorts for multiple patients, including the given patient, from the plurality of patients.

4. The method of claim 1, wherein:
the disease codes comprise International Classification of Diseases (ICD) codes,
the drug codes comprise National Drug Code (NDC) codes, and
the insurance codes comprise Healthcare Common Procedure Coding System (HCPCS) codes.

5. The method of claim 4, wherein:
identifying, based on the one or more disease codes in the medical office visit data or the medical insurance transaction data, the one or more biological conditions for the given patient comprise: identifying that the given patient has lung cancer based on ICD codes associated with lung cancer.

6. The method of claim 5, wherein:
identifying, based on the one or more disease codes in the medical office visit data or the medical insurance transaction data, the metastatic state for the given patient is based on a secondary malignancy ICD code or HCPCS code.

7. The method of claim 1, wherein the primary diagnosis biological condition for the given patient is determined based on disease codes, drug codes or insurance codes associated with a date within a predefined date range.

8. The method of claim 1, wherein assigning the given patient to the cohort comprises:
analyzing one or more data tables that include medical insurance transaction data for the given patient, the one or more data tables being from the one or more medical data repositories;
determining one or more first insurance transactions that include one or more first code identifiers included in the one or more data tables, wherein the one or more first code identifiers correspond to diagnoses of the patient with respect to the one or more biological conditions;
determining one or more second insurance transactions that include one or more second code identifiers included in the one or more data tables, wherein the one or more second code identifiers corresponding to medical procedures administered with respect to the patient, the medical procedures being from the medical office visit data;
generating a medical headers table that includes a first number of columns storing the one or more first code identifiers, a second number of columns storing the one or more second code identifiers, and a plurality of rows with individual rows of the plurality of rows corresponding to a first medical insurance transaction of the one or more first insurance transactions or a second medical insurance transaction of the one or more second insurance transactions;
storing the medical headers table in the one or more medical data repositories; and
determining the cohort for the patient based on data in the medical headers table.

9. The method of claim 8, wherein:
individual first insurance transactions of the one or more first insurance transactions indicate a date of service of the individual first insurance transactions; and
individual second insurance transactions of the one or more second insurance transactions indicate a date of service of the individual second insurance transactions.

10. The method of claim 9, further comprising:
arranging the plurality of rows of the medical headers table in ascending order based on the dates of service such that a claim with an earliest date of service is a first row of the medical headers table and that a transaction with a most recent date of service is a last row of the medical headers table.

11. The method of claim 8, further comprising:
analyzing the one or more first code identifiers to determine that a first code identifier of the one or more first code identifiers is included in a group of insurance code identifiers that correspond to the one or more biological conditions.

12. The method of claim 11, wherein:
the first code identifier is arranged according to a first format that corresponds to a first classification of insurance code identifiers;
the first classification of insurance code identifiers corresponds to International Classification of Diseases version 9 (ICD-9).

13. The method of claim 11, wherein:
the first code identifier is arranged according to a second format that corresponds to a second classification of insurance code identifiers;
the second classification of insurance code identifiers corresponds to International Classification of Diseases version 10 (ICD-10).

14. The method of claim 11, wherein:
the one or more biological conditions include a plurality of subtypes;
individual subtypes of the plurality of subtypes correspond to a subset of the group of insurance code identifiers that correspond to the one or more biological conditions; and
the method further comprises determining that the first code identifier is included in a first subset of the group of insurance codes that corresponds to a first subtype of the biological condition.

15. The method of claim 11, wherein the biological condition is cancer and the plurality of subtypes include at least one of lung cancer, breast cancer, or colorectal cancer.

16. The method of claim 15, further comprising:
determining one or more third insurance transactions having a date of service that is within a predefined time period ending on a first date of service;
analyzing one or more third insurance code identifiers of the third insurance transactions with respect to the first code identifiers and the second code identifiers.

17. The method of claim 16, further comprising:
determining that the one or more third insurance code identifiers are not included in the first code identifiers and the second code identifiers; and
determining, based on the third insurance code identifiers, that the patient is included in a cohort of patients in which a given subtype of the one or more biological conditions is present.

18. The method of claim 16, wherein the one or more third insurance code identifiers correspond to an additional biological condition.

19. The method of claim 16, further comprising:
determining that the one or more third insurance code identifiers are included in a portion of the group of insurance code identifiers that are not included in the subset of the group of insurance code identifiers;
determining that a date of service of at least one of the one or more third insurance transactions is a same date as the date of service as one of the one or more first insurance transactions;
determining that there are no other additional insurance transactions having insurance code identifiers included in the group of code identifiers; and
determining that the patient is included in a cohort of patients in which the subtype of the biological condition is present.

20. The method of claim 16, further comprising:
determining that the one or more third insurance code identifiers are included in a portion of the group of insurance code identifiers that are not included in the subset of the group of insurance code identifiers;
determining that a date service of at least one of the one or more third insurance claim is prior to the date of service of one of the one or more first insurance transactions and within the predefined time period; and
determining that the patient is not included in a cohort of patients in which the subtype of the biological condition is present.

21. A method implemented at one or more computing machines comprising processing circuitry and memory, the method comprising:
accessing, at the processing circuitry, one or more medical data tables storing medical insurance transaction data for a plurality of patients, the one or more medical data tables comprising a date column and a diagnosis column;
identifying, using the processing circuitry and based on the diagnosis column, a set of patients having a specified biological condition, the set of patients being from among the plurality of patients;
determining, for each patient in the set of patients, an earliest date when the patient received a diagnosis of the specified biological condition;
identifying, using the processing circuitry and based on the diagnosis column and the date column, a cohort of patients from among the set of patients, the cohort of patients lacking a diagnosis from a collection of biological conditions associated with a date occurring during a predefined time window before the earliest date when the patient received the diagnosis of the specified biological condition; and
providing an output representing the cohort.

22. The method of claim 21, wherein the diagnosis column stores International Classification of Diseases version 9 (ICD-9) or International Classification of Diseases version 10 (ICD-10) codes.

23. The method of claim 21, wherein the specified biological condition is lung cancer, wherein the collection of biological conditions comprises cancers different from lung cancer, wherein the predefined time window before the earliest date is six months before the earliest date.

24. The method of claim 21, wherein the specified biological condition is a specified type of cancer, the method further comprising:
determining a metastatic state of at least one patient from the cohort.

25. The method of claim 24, wherein the metastatic state is determined based on a secondary malignancy International Classification of Diseases (ICD) code or Healthcare Common Procedure Coding System (HCPCS) code.

26. The method of claim 21, wherein identifying the cohort comprises:
arranging rows associated with patients in the set by date; and
accessing rows associated with the predefined time window to identify patients in the set that lack the diagnosis from the collection of biological conditions during the predefined time window.

27-52. (canceled)

53. A system comprising:
processing circuitry; and
a memory storing instructions which, when executed by the processing circuitry, cause the processing circuitry to perform operations comprising:
accessing, at the processing circuitry, one or more medical data repositories storing data for a given patient from among a plurality of patients, the one or more medical data repositories storing pharmacy data, medical office visit data, and medical insurance transaction data;
identifying, based on one or more disease codes in the medical office visit data or the medical insurance transaction data, one or more biological conditions and a metastatic state for the given patient;
identifying, based on one or more drug codes in the pharmacy data, one or more lines of treatment for the given patient;
identifying, based on one or more insurance codes in the medical insurance transaction data, one or more medical procedures undergone by the given patient;
determining, based on a combination of the one or more biological conditions, the metastatic state, the one or more lines of treatment, and the one or more medical procedures, a primary diagnosis biological condition for the given patient;
assigning, based on the primary diagnosis biological condition, the given patient to a cohort of patients; and
providing an output representing the assigned cohort for the given patient.

54-93. (canceled)
